(19) United States
(12) Patent Application Publication
(10) Pub. No.: US 2024/0374572 A1
(43) Pub. Date: Nov. 14, 2024

(54) METHODS FOR EXTENDING THE LIFESPAN OF CELLS AND ORGANISMS

(71) Applicant: Ergo-Health LLC, Auburndale, MA (US)

(72) Inventor: Pinghua Liu, Newton, MA (US)

(73) Assignee: Ergo-Health LLC, Auburndale, MA (US)

(21) Appl. No.: 18/437,501

(22) Filed: Feb. 9, 2024

Related U.S. Application Data
(63) Continuation-in-part of application No. PCT/US22/74723, filed on Aug. 9, 2022.
(60) Provisional application No. 63/231,080, filed on Aug. 9, 2021.

(51) Int. Cl. A61K 31/4172; A61P 39/00

(52) U.S. Cl. CPC A61K 31/4172; A61P 39/00

(57) ABSTRACT

This disclosure provides a method for modulating the lifespan of a cell, tissue, organ or organism, comprising contacting the cell, tissue, organ or organism with a composition comprising an effective amount of ergothioneine, or an analog, a derivative, or a pharmaceutically acceptable prodrug, a pharmaceutically active metabolite, a pharmaceutically acceptable salt thereof. Also provided is a method of treating a subject having a premature ageing disease or condition.

### CROSS-REFERENCE TO RELATED APPLICATIONS

The present application is a continuation-in-part of International Patent Application No. PCT/US22/74723, filed Aug. 9, 2022, which claims the benefit of priority under 35 U.S.C. § 119 (e) to U.S. Provisional Patent Application No. 63/231,080, filed Aug. 9, 2021. The foregoing applications are incorporated by reference herein in their entireties.

### STATEMENT REGARDING FEDERALLY SPONSORED RESEARCH OR DEVELOPMENT

Subject matter disclosed herein was supported by the National Science Foundation grant number CHE-130914. The U.S. government has certain rights in the claimed invention.

### REFERENCE TO A SEQUENCE LISTING

This application contains a Sequence Listing which has been submitted electronically in xml format and is hereby incorporated by reference in its entirety. Said xml copy, created on May 23, 2024, is named “Seq Listing 15792400018.xml” and is 18,493 bytes in size.

### FIELD OF THE INVENTION

This invention relates generally to methods and reagents for treating ageing related diseases or conditions.

### BACKGROUND OF THE INVENTION

According to the World Health Organization, the global average life expectancy was 31 years in 1900 and increased to 72 years in 2015. Given the global ageing population, age-related conditions are the major burden of our health-care system. Therefore, delaying the onset of ageing related diseases will afford significant and numerous medical and financial benefits. It has been suggested that delaying ageing by 2.2 years will result in $7 trillion in savings over 50 years for the US healthcare system. Age-related disorders including Alzheimer's disease affect an estimated 4.5 million Americans. National direct and indirect annual costs of caring for individuals with Alzheimer's disease are at least $100 billion, according to estimates used by the Alzheimer's Association and the National Institute on Ageing. Parkinson's disease affects over 1 million people in the US alone and is one of the most common debilitating diseases in the country. According to the National Parkinson's Foundation, each individual spends an average of $2,500 a year for medications. Estimates of costs of medical care, disability payments and lost income exceed $5.6 billion annually. Thus, age-related disorders have a significant impact and understanding the ageing process may help develop new therapies for these and other disorders.

All living cells and organisms have a finite lifespan. They live for a period of time and die. Cells and organisms have both a chronological age and a biological age. The former is measured in days, months, or years while the latter may be measured by a host of complex testing of biological functions including but not limited to gene expression, protein production or metabolic pathways. The rate of ageing may also be measured, and an accelerated rate of ageing may be considered ‘premature ageing,’ while a slower rate of ageing may extend lifespan. It is desirable to maximize the healthy lifespan of cells and organisms, and it is also desirable to extend the healthy lifespan by delaying the rate of ageing and the onset of dysfunctional or disease states.

Thus, there remains a strong need for developing methods and reagents for slowing the ageing process and extending the lifespan of cells and organisms.

### SUMMARY OF THE INVENTION

This disclosure addresses the need mentioned above in a number of aspects. In one aspect, this disclosure provides a method for modulating the lifespan of a cell, tissue, organ or organism, comprising contacting the cell, tissue, organ or organism with a composition comprising an effective amount of a compound of Formula I, or a pharmaceutically acceptable prodrug, a pharmaceutically active metabolite, a pharmaceutically acceptable salt thereof, wherein the compound is represented by:

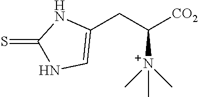

In some embodiments, the method includes increasing the level of activity of a gene, such as increasing the expression level of the gene. The gene may have a nucleotide sequence of SEQ ID NOs.: 1-5. In some embodiments, the gene may be one of DAF-16, FOXO1, FOX02, FOXO3, and FOX06.

In some embodiments, the method may include down-regulating any one of RAB-1, PMK-1, AKT-1, AKT2, JNK-1, PDK-1, and DAF-2.

In some embodiments, the method further includes contacting the cell with a second agent (e.g., rapamycin).

In another aspect, this disclosure provides a method for increasing the level of activity of a gene selected from the group consisting of DAF-16, FOXO1, FOXO2, FOXO3, and FOX06 in a cell, tissue, organ or organism, comprising contacting the cell, tissue, organ or organism with a composition comprising an effective amount of ergothioneine, or an analog, a derivative, or a pharmaceutically acceptable prodrug, a pharmaceutically active metabolite, a pharmaceutically acceptable salt thereof.

In yet another aspect, the disclosure provides a method for modulating response or resistance to stress of a cell, tissue, organ or organism, comprising contacting the cell, tissue, organ or organism with a composition comprising an effective amount of the compound described herein, or an analog, a derivative, or a pharmaceutically acceptable prodrug, a pharmaceutically active metabolite, a pharmaceutically acceptable salt thereof.

In some embodiments, the cell exists in an in vitro condition. The cell can be a mammalian cell or a plant cell. In some embodiments, the cell can be an autologous cell or an allograft cell. In some embodiments, the cell can be an embryo or in vitro fertilization cell. In some embodiments, the cell can be a eukaryotic cell or a prokaryotic cell. In some embodiments, the cells can be keratinocytes, fibroblasts, melanocytes, endothelial cells, Langerhans cells, Merkel cells, adipocytes, nerve cells, hair, sweat, oil, stem cells or muscle cells.

In some embodiments, the organism is a mammal, and the composition can be administered intratumorally, intravenously, subcutaneously, intraosseously, orally, transdermally, in sustained release, in controlled release, in delayed release, as a suppository, or sublingually.

Another aspect provides a method of treating a subject diagnosed to have a premature ageing disease or condition. In some embodiments, the method comprises treating the subject with a therapeutically effective amount of compound I or a pharmaceutical composition thereof. In some embodiments, the premature ageing disease is Hutchinson-Gilford progeria syndrome. In some embodiments, the premature ageing disease or condition is premature ageing of skin, mucous membranes, scalp and/or hair of the subject.

In some embodiments of any of the above-described aspects, the composition or compound can be administered daily. In some embodiments, the composition may include a pharmaceutically acceptable carrier. In some embodiments, the compound or pharmaceutically acceptable salt thereof has a purity of above about 90%.

In some embodiments, a dosage of between about 5 mg/kg and about 200 mg/kg of the composition is administered the mammal.

In another aspect, this disclosure provides a method of up-regulating a Klk1-related Kallikrein system. In some embodiments, the method comprises administering a subject a compound represented by the formula below:

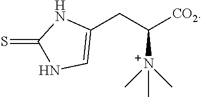

wherein the administration of the compound up-regulates Klk1 by at least 100%.

In another aspect, this disclosure provides a method of reducing a symptom of stroke in a subject in need thereof. In some embodiments, the method comprises administering the subject a compound represented by the formula below:

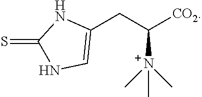

wherein the administration of the compound reduces oxygen glucose deprivation in a central nervous system of the subject.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIGS. 1A, 1B, and 1C are schematic representations of the proposed biosynthetic pathways for ergothioneine.

FIGS. 2A, 2B, 2C, and 2D show the life-extension effect of ergothioneine.

FIGS. 3A, 3B, and 3C show the effect of ergothioneine in daf-16, akt-2, and skn-1 mutants, respectively.

FIG. 4 shows the accelerated aging of mouse model.

FIGS. 5A, 5B, 5C, and 5D show the animal study in rats (FIG. 5A: untreated disease group; FIG. 5B treated disease group). FIG. 5C shows the life span of mice with and without ergothioneine. FIG. 5D shows oxygen glucose deprivation for primary cortical neuron culture.

FIG. 6 shows the physical activities of untreated disease group and treated disease group.

FIGS. 7A, 7B, 7C, and 7D show the protective effect of ergothioneine on stroke MCAO model (images of tissue sample (FIG. 7A), infarct volume (FIG. 7B), neurological deficit (FIG. 7C), and cerebral blood flow (CBF) (FIG. 7D) of Sham, vehicle, and ergothioneine treated mice).

FIGS. 8A and 8B that the results of RNA-seq analysis of peri-infarct area of the MCAO model. FIG. 8A shows the RNA levels of various gene including KLK1.

### DETAILED DESCRIPTION OF THE INVENTION

The disclosed methods for modulating (e.g., extending) the lifespan of a cell, tissue, organ or organism are based, at least in part, on an unexpected discovery that ergothioneine and its analogs or variants have life-extension effect on a worm model under regular culture conditions and under stresses (e.g., oxidative stress conditions, heat-shock conditions). This disclosure demonstrates that ergothioneine exert its life-extension effect through the daf-16 related signaling pathway. The discovery of ergothioneine's life-extension effect and the demonstration of the involvement of daf-16 opens the door for more systematic evaluation for ergothioneine's protective effect on various chronic disease animal models. Results from animals studies further confirm the therapeutic effect of Compound I.

### Methods for Extending Lifespan

This disclosure provides a method for modulating the lifespan of a cell, tissue, organ or organism, comprising contacting the cell, tissue, organ or organism with a composition comprising an effective amount of a compound of Formula I, or a pharmaceutically acceptable prodrug, a pharmaceutically active metabolite, a pharmaceutically acceptable salt thereof, wherein the compound is represented by:

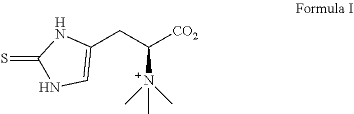

“Lifespan” refers to the length of time a cell, tissue or organism remains viable. The potential or inherent lifespan is defined as the unaltered lifespan of the cell or organism based solely on genetic factors, and the observed lifespan is defined as the length of time the cell or organism will remain viable when all damaging stimuli (e.g., oxidative stress, poor nutrition) are factored in. The life span of cells can be increased by at least about 20%, 30%, 40%, 50%, 60% or between 20% and 70%, 30% and 60%, 40 and 60% or more using the methods of the invention.

In any of the provided methods, the method can take place in a cell that is in an in vitro condition. Optionally, the cell is a mammalian cell (e.g., cells are selected from keratinocytes, fibroblasts, melanocytes, endothelial cells, Langerhans cells, Merkel cells, adipocytes, nerve cells, hair, sweat, oil, stem cells and/or muscle cells), a plant cell, a microbial cell, a stem cell, an autologous cell or an allograft cell, an embryo or in vitro fertilization cell. In different embodiments, the cell is a eukaryotic cell or a prokaryotic cell.

Methods herein apply to extend the health and lifespan of human cells (and tissues, organs, and organisms), as well as cells (and tissues, organism, and organisms) of non-human animals, unicellular and multicellular organisms, plants, and so forth. Thus, it will be understood when a gene is referred to, that reference includes the orthologous sequence(s) from other species, etc.

### Modulating Activity and/or Expression Level of Genes Implicated in Ageing

In some embodiments, the method includes increasing the level of activity of a gene, such as increasing the expression level of the gene. The gene may have a nucleotide sequence of SEQ ID NOs.: 1-5. In some embodiments, the gene may be one of DAF-16, FOXO1, FOXO2, FOXO3, and FOX06.

In some embodiments, the method may include down-regulating any one of RAB-1, PMK-1, AKT-1, AKT-2, JNK-1, PDK-1, and DAF-2.

In another aspect, this disclosure provides a method for increasing the level of activity of a gene selected from the group consisting of DAF-16, FOXO1, FOXO2, FOXO3, and FOX06 in a cell, tissue, organ or organism, comprising contacting the cell, tissue, organ or organism with a composition comprising an effective amount of ergothioneine, or an analog, a derivative, or a pharmaceutically acceptable prodrug, a pharmaceutically active metabolite, a pharmaceutically acceptable salt thereof.

TABLE 1
Genes implicated in a ageing process

| GENE | ACCESSION CODE | SEQ ID NO |
|---|---|---|
| DAF-16 | NM_001313505.1 | SEQ ID NO: 1 |
| FOXO1 | NP_002006.2 | SEQ ID NO: 2 |
| FOXO2 | NM_201559.2 | SEQ ID NO: 3 |
| FOXO3 | NM_001455.4 | SEQ ID NO: 4 |
| FOXO6 | NM_001291281.3 | SEQ ID NO: 5 |
| RAB-1 | AFP33144.1 | SEQ ID NO: 6 |
| PMK-1 | CCD61386.1 | SEQ ID NO: 7 |
| AKT-1 | CAD44085.1 | SEQ ID NO: 8 |
| AKT-2 | CAC70087.1 | SEQ ID NO: 9 |
| JNK-1 | NP_079504.2 | SEQ ID NO: 10 |
| PDK-1 | NP_002602.2 | SEQ ID NO: 11 |
| DAF-2 | AAC47715.1 | SEQ ID NO: 12 |

TABLE 2
Exemplary sequences

| SEQ ID | | NOTE |
|---|---|---|
| NO | SEQUENCE | |
| SEQ ID | MMEMLVDQGTDASSSASTSTSSVSR | DAF-16 |
| NO: 1 | FGADTFMNTPDDVMMNDDMEPIPRD | Accession |
| | RCNTWPMRRPQLEPPLNSSPIIHEQ | code: |
| | IPEEDADLYGSNEQCGQLGGASSNG | NM_001313 |
| | STAMLHTPDGSNSHQTSFPSDFRMS | 505.1 |
| | ESPDDTVSGKKTTTRRNAWGNMSYA | |
| | ELITTAIMASPEKRLTLAQVYEWMV | |
| | QNVPYFRDKGDSNSSAGWKNSIRHN | |
| | LSLHSRFMRIQNEGAGKSSWWVINP | |
| | DAKPGRNPRRTRERSNTIETTTKAQ | |
| | LEKSRRGAKKRIKERALMGSLHSTL | |
| | NGNSIAGSIQTISHDLYDDDSMQGA | |
| | FDNVPSSFRPRTQSNLSIPGSSSRV | |
| | SPAIGSDIYDDLEFPSWVGESVPAI | |
| | PSDIVDRTDQMRIDATTHIGGVQIK | |
| | QESKPIKTEPIAPPPSYHELNSVRG | |
| | SCAQNPLLRNPIVPSTNFKPMPLPG | |
| | AYGNYQNGGITPINWLSTSNSSPLP | |
| | GIQSCGIVAAQHTVASSSALPIDLE | |
| | NLTLPDQPLMDTMDVDALIRHELSQ | |
| | AGGQHIHFDL | |
| SEQ ID | MAEAPQVVEIDPDFEPLPRPRSCTW | FOXO1 |
| NO: 2 | PLPRPEFSQSNSATSSPAPSGSAAA | Accession |
| | NPDAAAGLPSASAAAVSADFMSNLS | code: |
| | LLEESEDFPQAPGSVAAAVAAAAAA | NP_002006.2 |
| | AATGGLCGDFQGPEAGCLHPAPPQP | |
| | PPPGPLSQHPPVPPAAAGPLAGQPR | |
| | KSSSSRRNAWGNLSYADLITKAIES | |
| | SAEKRLTLSQIYEWMVKSVPYFKDK | |
| | GDSNSSAGWKNSIRHNLSLHSKFIR | |
| | VQNEGTGKSSWWMLNPEGGKSGKSP | |
| | RRRAASMDNNSKFAKSRSRAAKKKA | |
| | SLQSGQEGAGDSPGSQFSKWPASPG | |
| | SHSNDDFDNWSTFRPRTSSNASTIS | |
| | GRLSPIMTEQDDLGEGDVHSMVYPP | |
| | SAAKMASTLPSLSEISNPENMENLL | |
| | DNLNLLSSPTSLTVSTQSSPGTMMQ | |
| | QTPCYSFAPPNTSLNSPSPNYQKYT | |
| | YGQSSMSPLPQMPIQTLQDNKSSYG | |
| | GMSQYNCAPGLLKELLTSDSPPHND | |
| | IMTPVDPGVAQPNSRVLGQNVMMGP | |
| | NSVMSTYGSQASHNKMMNPSSHTHP | |
| | GHAQQTSAVNGRPLPHTVSTMPHTS | |
| | GMNRLTQVKTPVQVPLPHPMQMSAL | |
| | GGYSSVSSCNGYGRMGLLHQEKLPS | |
| | DLDGMFIERLDCDMESIIRNDLMDG | |
| | DTLDFNFDNVLPNQSFPHSVKTTTH | |
| | SWVSG | |

Transcription levels can be quantitated absolutely or relatively. Absolute quantitation can be accomplished by inclusion of known concentrations of one or more target nucleic acids (for example control nucleic acids or with a known amount the target nucleic acids themselves) and referencing the hybridization intensity of unknowns with the known target nucleic acids (for example by generation of a standard curve).

The terms “decrease,” “reduced,” “reduction,” “decrease” or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

DAF-16 is the sole ortholog of the FOXO family of transcription factors in the nematode Caenorhabditis elegans. It is responsible for activating genes involved in longevity, lipogenesis, heat shock survival, and oxidative stress responses. It also protects C. elegans during food deprivation, causing it to transform into a hibernation-like state, known as a Dauer. DAF-16 is notable for being the primary transcription factor required for the profound lifespan extension observed upon mutation of the insulin-like receptor DAF-2. The gene has played a large role in research into longevity and the insulin signaling pathway as it is located in (C. elegans, a successful ageing model organism.

The C. elegans insulin/IGF-1-like signaling pathway is the best-characterized pathway modulating ageing, and single gene mutations in this pathway have been shown to significantly affect lifespan. For example, loss of function mutations in daf-2, the insulin/IGF-1 receptor homolog, cause the animal to live twice as long as wild-type animals (Kenyon, C. et al., (1993) Nature, 366 (6454), 461-4). Many molecular studies have shown that DAF-2 activates a phosphatidylinositol-3-OH (PI3K) signaling cascade, which ultimately acts to antagonize the DAF-16 forkhead family transcription factor. DAF-16 is then unable to repress genes that regulate longevity, as well as the dauer diapause decision and stress response, and to activate genes that are required for metabolism and reproductive growth.

DAF-16 is conserved across species, with homologs being found in (C. elegans, humans, mice, and Drosophila (fruit flies). In C. elegans, DAF-16 is located on Chromosome 1, at position 175-268. It is made up of 15 exons. DAF-16 is also located downstream of DAF-2, which signals in the IIS pathway. Mutants in this pathway age slower and have a lifespan up to twice as long as normal. Further studies have demonstrated that the lifespan extension is dependent on DAF-16. Other consequences of mutations in the DAF-16 gene is the inability to form dauers.

DAF-16 encodes FOXO (Forkhead box protein O), which binds to gene promoters that contain the sequence TTGTTTAC in their regulatory region—this is the DAF-16 binding element (DBE). FOXO is involved in the Insulin/IGF1 signaling pathway (IIS) which affects longevity, lipogenesis, dauer formation, heat shock, and oxidative stress responses, by activating proteins such as MnSOD and Catalase. Expression of FOXO in the intestine normally leads to longevity signaling. FOXO has been shown to have a protective role against cancer, as it regulates and suppresses genes involved in tumor formation. It also has a protective role against muscular dystrophy. FOXO is also important in embryonic development, as it promotes apoptosis.

FOX (Forkhead box) proteins are a family of transcription factors that play important roles in regulating the expression of genes involved in cell growth, proliferation, differentiation, and longevity. Many FOX proteins are important to embryonic development. FOX proteins also have pioneering transcription activity by being able to bind condensed chromatin during cell differentiation processes. The defining feature of FOX proteins is the forkhead box, a sequence of 80 to 100 amino acids forming a motif that binds to DNA. This forkhead motif is also known as the winged helix due to the butterfly-like appearance of the loops in the protein structure of the domain. Forkhead proteins are a subgroup of the helix-turn-helix class of proteins.

Many genes encoding FOX proteins have been identified. For example, the FOXF2 gene encodes forkhead box F2, one of many human homologs of the Drosophila melanogaster transcription factor forkhead. FOXF2 is expressed in lung and placenta. Some FOX genes are downstream targets of the hedgehog signaling pathway, which plays a role in the development of basal cell carcinomas. Members of the class O regulate metabolism, cellular proliferation, stress tolerance, and possibly lifespan. The activity of FOXO is controlled by post-translational modifications, including phosphorylation, acetylation, and ubiquitination.

In yet another aspect, the disclosure provides a method for modulating response or resistance to stress (e.g., oxidative stress, heat shock, nutrient or calorie deprivation) of a cell, tissue, organ or organism, comprising contacting the cell, tissue, organ or organism with a composition comprising an effective amount of the compound of Formula I, or a pharmaceutically acceptable prodrug, a pharmaceutically active metabolite, a pharmaceutically acceptable salt thereof.

Oxidative stress refers to an imbalance within the cell, tissue or organism which results in a diminished ability to: reduce (or detoxify) biological reactive chemical intermediates, repair the damage caused by reactive chemical intermediates, or maintain the cellular reduction potential most often resulting in apoptosis.

Any of the above-described methods may further include a step of identifying or determining the cell, tissue, organ or organism as having premature ageing disease or condition. Procedures of the determination step for premature ageing disease or condition are generally known in the field.

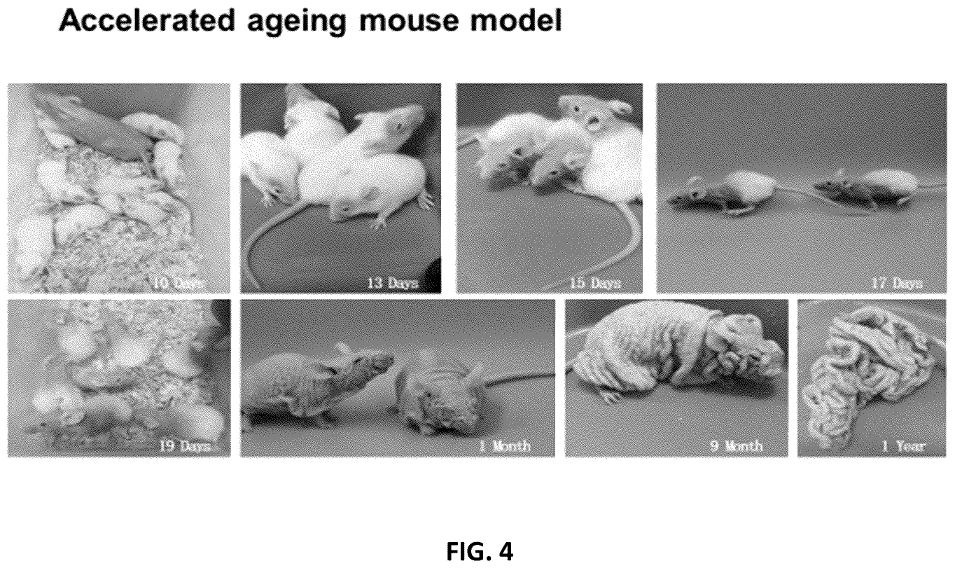

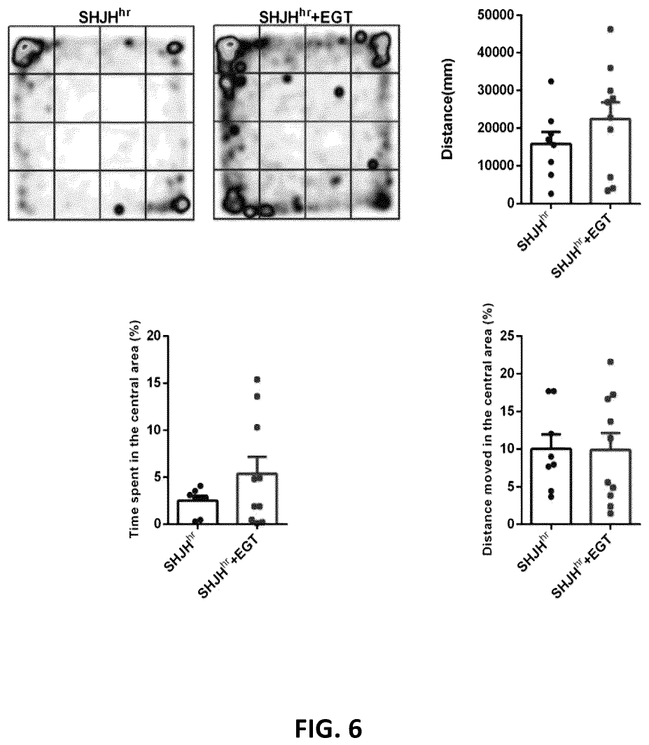

Another aspect provides a method of treating a subject diagnosed to have a premature ageing disease or condition, comprising treating the subject with a therapeutically effective amount of compound I. In some embodiments, the premature ageing disease is Hutchinson-Gilford progeria syndrome. In some embodiments, the premature ageing disease is Werner syndrome. In some embodiments, the premature ageing disease or condition is premature ageing of skin, mucous membranes, scalp and/or hair of the subject. In some embodiments, the method may further include a step of identifying or determining the subject as having premature ageing disease or condition. Procedures of the determination step for premature ageing disease or condition are generally known in the field. As shown in the example section and FIGS. 4-6, the study (accelerated ageing mouse model) analyzed the physical appearance and activities of two groups (with and without the feeding of Compound I, 20 mg/kg daily) over the period of about 1 year. The group treated with compound 1 have higher average weight and more smooth skin condition than the other group without the treatment. The group without any treatment were also less active physically. These results indicate that compound I could effectively slow down the ageing process.

In the methods described herein, the compound of Formula I or a pharmaceutically acceptable prodrug, a pharmaceutically active metabolite, a pharmaceutically acceptable salt thereof is administered in an effective amount to achieve the desired effect. In some embodiments, the compound whether in salt form or salt-free form, is administered at more than about 1, more than about 2, more than about 5, more than about 10, more than about 15, more than about 20, more than about 25, more than about 50, more than about 100, more than about 200, or more than about 500 mg/kg/day to a subject in need. The subject is an animal or a human. In some embodiments, the compound or a pharmaceutically acceptable salt thereof being used or administered has a purity of above about 90%, above about 95%, above about 98%, or above about 99%. In some embodiments, the subject is administered Compound I at a daily dosage ranging from about 1 mg to about 1000 mg, from about 2 mg to about 800 mg, from about 5 mg to about 500 mg, from about 5 mg to about 200 mg, from about 10 mg to about 200 mg, from about 10 mg to about 100 mg, from about 50 mg to about 500 mg, from about 50 mg to about 100 mg, or from about 100 mg to about 200 mg.

The compound can be administered intratumorally, intravenously, subcutaneously, intraosseously, orally, transdermally, in sustained release, in controlled release, in delayed release, as a suppository, or sublingually. In some embodiments, the method further includes administering to the subject a second agent selected from the group consisting of Nox1 inhibitor, PI3K/AKT/mTOR pathway inhibitors, hydroxymethylglutaryl-coenzyme A (HMG-CoA) reductase inhibitor. In some embodiments, the second agent is rapamycin.

### Protective Effects on Stroke

In another aspect, this disclosure provides a method of up-regulating a Klk1-related Kallikrein system. In some embodiments, the method comprises administering a subject a compound represented by the formula below:

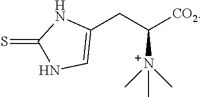

wherein the administration of the compound up-regulates Klk1 by at least 100%.

In another aspect, this disclosure provides a method of reducing a symptom of stroke in a subject in need thereof. In some embodiments, the method comprises administering the subject a compound represented by the formula below:

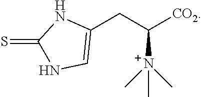

wherein the administration of the compound reduces oxygen glucose deprivation in a central nervous system of the subject.

### Ergothioneine, Variants Thereof, and Other Compounds

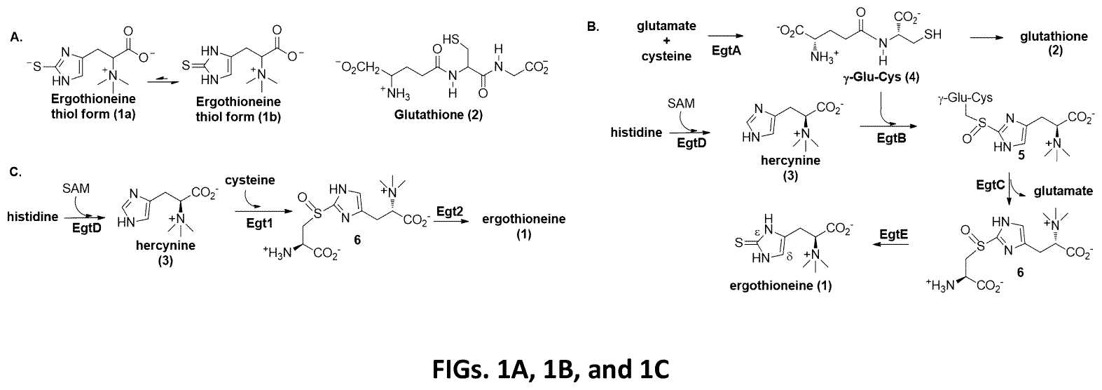

Compound I (Ergothioneine) is a thiol-histidine derivative (see also FIG. 1A). Glutathioneine is biosynthesized by animals themselves. In contrast, animals do not have the capacity to synthesize ergothioneine, and all of their ergothioneine is obtained from their food. Through an ergothioneine-specific transporter OCTN1, ergothioneine accumulates up to 2 mM in many parts of the body with the highest concentration in erythrocytes, bone marrow, liver, kidney, seminal fluid and the lens and cornea of the eyes. Several human diseases have been suggested to be related to ergothioneine insufficiency.

However, ergothioneine biological function evaluation faces two key barriers. First, the current ergothioneine chemical synthetic method is very challenging, which makes it prohibitively expensive to researchers to conduct mechanistic studies. Second, its biological targets were not known. Recently, our laboratory developed the first fermentation-based ergothioneine production method.

Among natural thiols, ergothioneine is unique because it is present predominantly in the form of thione. As a result, ergothioneine's reduction potential (E0′=−0.06 V) is significantly higher than that of glutathione (E0′=−240 mV). Because ergothioneine and glutathioneine are among the most abundant natural thiols in animals, a combination of these two thiols offers cellular protection over a wide range of oxidative stress conditions. Ergothioneine insufficiency has been suggested to be related to rheumatoid arthritis, Crohn's disease, and diabetes.

Ergothioneine can pass through the blood-brain barrier and may have protective effects against neurodegenerative diseases. Ergothioneine treatment decreases the expression of adhesion molecules VACM-1, ICAM-1, and E-selectin, which may be responsible for ergothioneine's protective role against cardiovascular disorders. Recently, ergothioneine has also been explored as a protective reagent in organ-transplant.

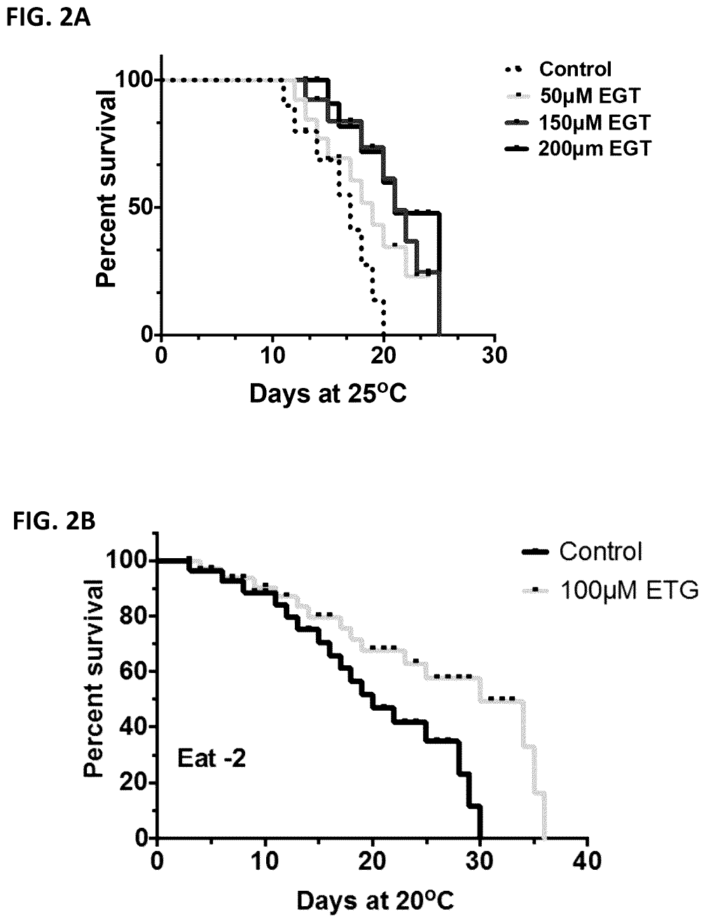

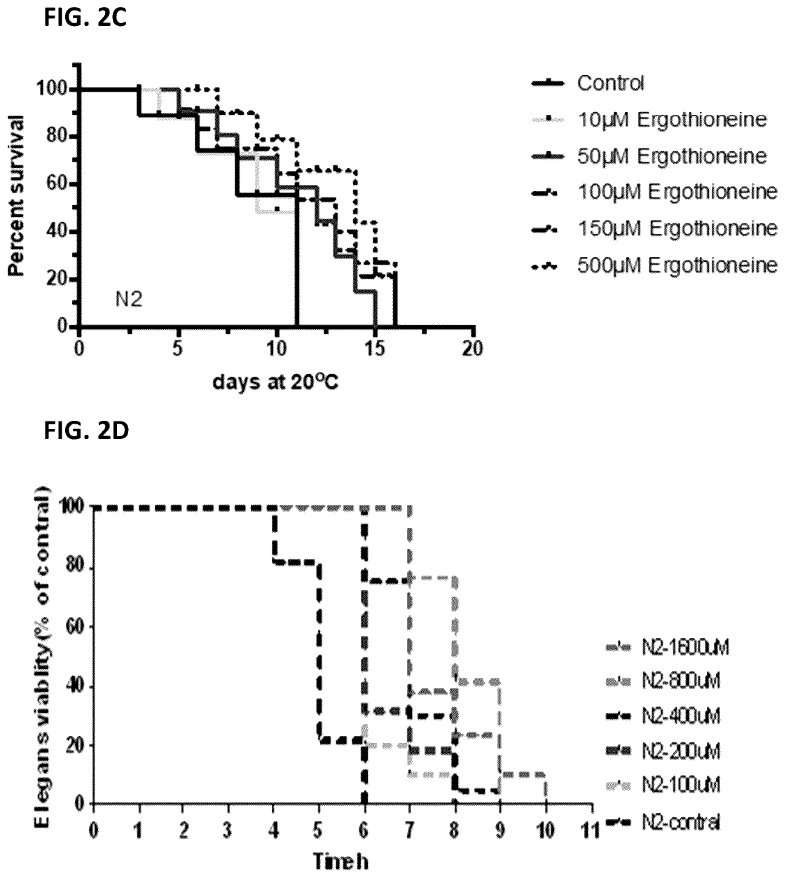

Despite many of these beneficial roles to human health, the ergothioneine chemical synthesis reported in 1995 remains as the only industrial ergothioneine production method. Due to the difficulties faced in some of the steps, ergothioneine is not easily accessible to scientists for detailed functional characterizations. Ergothioneine biosynthesis was actively pursued for decades, and a main breakthrough was achieved until recently. Seebeck and co-workers discovered the ergothioneine biosynthetic gene cluster in Mycobacterium smegmatis (FIG. 1B), designated egtABCDE (EgtA-EgtE genes). In this biosynthetic process, there are two key steps, a mononuclear non-heme iron enzyme (EgtB) catalyzed oxidative coupling between hercynine and γ-glutamylcysteine (γ-Glu-Cys) and a PLP-dependent C-S lyase (EgtE). Because γ-Glu-Cys (4) is also the biosynthetic intermediate for glutathione biosynthesis (FIG. 1B), such competition severely limits the ergothioneine production yield when the M. smegmatis biosynthetic pathway was applied to produce ergothioneine using fermentation. To address this challenge, the inventors demonstrated that the fungal Neurospora crassa non-heme iron enzyme Egt1 makes use of hercynine and cysteine as the substrate (FIG. 2C). After reconstitution of the N. crassa ergothioneine biosynthetic pathway in E. coli, a yield of 1g/L can be readily achieved.

The production of ergothioneine through either in vitro enzymatic transformations or fermentations using microbials created by metabolic engineering is described in more detail in the U.S. patent No. U.S. Pat. No. 10,167,490B2, hereby incorporated in its entirety. For example, ergothioneine can be produced in an in vitro cell-free system comprising recombinantly expressed one or more genes of the ergothioneine biosynthetic pathway and one or more genes of the ovothiol biosynthetic pathway or their homologs identified through bioinformatics approach (e.g., NcEgt1).

In one example, a method for producing ergothioneine comprises: (i) incubating histidine or hercynine with a reaction mixture comprising recombinantly expressed EgtA protein or a functional fragment thereof retaining enzymatic activity, EgtB protein or a functional fragment thereof retaining enzymatic activity, EgtC protein or a functional fragment thereof retaining enzymatic activity, EgtD protein or a functional fragment thereof retaining enzymatic activity, and EgtE protein or a functional fragment thereof retaining enzymatic activity; and (ii) isolating ergothioneine from the enzymatic mixture.

In another example, a method for preparing ergothioneine comprises: (i) incubating hercynine with a reaction mixture comprising recombinantly expressed: (a) OvoA protein, NcEgt1 protein or a functional fragment thereof retaining enzymatic activity and (b) EgtE protein of a functional fragment thereof retaining enzymatic activity; and (ii) isolating ergothioneine from the enzymatic mixture.

In yet another example, a method for preparing ergothioneine comprises: (i) incubating histidine with a reaction mixture comprising recombinantly expressed: (a) EgtD protein or a functional fragment thereof retaining enzymatic activity, (b) OvoA protein, NcEgt1 protein or a functional fragment thereof retaining enzymatic activity, and (c) EgtE protein or a functional fragment thereof retaining enzymatic activity; and (ii) isolating ergothioneine from the enzymatic mixture.

Alternatively, ergothioneine can be produced in a cell that recombinantly expresses: (i) a egtA gene; (ii) a egtB gene; (iii) a egtC gene; (iv) a egtD; and (iv) a egtE gene and a gene encoding FAD synthetase. The cell further expresses a gene encoding a SAM synthetase.

Ergothioneine can also be produced in a cell that recombinantly expresses: (i) an ovoA gene or a ncEgt-1 gene or their homologs identified by bioinformatics; and (ii) an egtE gene and a gene encoding FAD synthetase.

In another example, ergothioneine can be produced in a cell that recombinantly expresses: (i) an egtD gene; (ii) an ovoA gene or a ncEgt-1 gene; and (iii) an egtE gene and a gene encoding FAD synthetase.

Alternatively, ergothioneine can be produced in a cell that recombinantly expresses: (i) an egtD gene; (ii) an ovoA gene or a ncEgt-1 gene; and (iii) an egtE gene and a gene encoding FAD synthetase; and (iv) a gene encoding a SAM synthetase.

As used herein, the term “cell,” “host cell” or “cell line” is intended to refer to well-characterized homogenous, biologically pure populations of cells. These cells can be eukaryotic cells that are neoplastic or which have been “immortalized” in vitro by methods known in the art, as well as primary cells, or prokaryotic cells. Without limitation, the host cell or the cell line can be of mamalian, plant, insect, fungal (including yeast), or bacterial origin.

In some embodiments, the cell is a bacterial cell. In some further embodiments of this, the cell is E. coli or S. pyogenes. Exemplary strains of E. coli amenable to various aspects described herein include, but are not limited to, BL21(DE3), BL21(DE3)pLysS, BL21(DE3)pLysE, BL21(DE3)pLac1, BL21trxB(DE3), BL21trxB(DE3)pLysS, BLR(DE3), BLR(DE3)pLysS, AD494(DE3), AD494(DE3)pLysS, HMS174(DE3), HMS174(DE3)pLysS, HMS174(DE3)pLysE, Origami(DE3), Origami(DE3)pLysS, Origami(DE3)pLysE, Origami(DE3)pLacl, OrigamiB(DE3), OrigamiB(DE3)pLysS, OrigamiB(DE3)pLysE, OrigamiB(DE3)pLacl, Rosetta(DE3), Rosetta(DE3)pLysS, Rosetta(DE3)pLysE, Rosetta(DE3)pLacl, Tuner(DE3), Tuner(DE3)pLysS and Tuner(DE3)pLacl.

The polypeptide encoded by the ovoA gene (OvoA enzyme) is a 5-histidylcysteine sulfoxide synthase. OvoA is a mononuclear non-heme enzyme and catalyzes a four-electron oxidation process. OvoA was the first ovothiol biosynthetic enzyme as characterized from Erwinia tasmaniensis and Trypanosoma cruzi. Homologous Enzymes homologs OvoA are encoded in more than 80 genomes ranging from proteobacteria to fungi.

The polypeptide encoded by the ncEgt-1 gene (NcEgt-1 enzyme from Neurospora crassa) catalyzes the formation of ergothioneine from hercynine and cysteine.

The polypeptide encoded by the egtE gene (EgtE enzyme) catalyzes the cleavage of a C—S bond in the product of oxidative coupling between hercynine and cysteine.

The polypeptide encoded by the egtD gene (EgtD enzyme) enzyme catalyzes the N-methylation of histidine to produce hercynine.

As EgtD is a SAM-dependent methyltransferase, a SAM synthetase can be co-expressed with EgtD to enhance the activity of EgtD. Accordingly, in some embodiments, the cell can be further transformed to recombinantly express a gene encoding a SAM synthetase. The enzyme SAM synthetase (EC 2.5.1.6) catalyzes the conversion of methionine and ATP into S-adenosylmethionine (AdoMet or SAM). The genes for SAM synthetase, which catalyzes the conversion of methionine to SAM, have been cloned from E. coli.

Since SAM synthetase substrates are methionine and ATP, the cell culture media, for production of ergothioneine, can be further supplemented with methionine in addition to histidine or hercynine.

Expression of EgtE in a cell can be enhanced under a condition that has FAD synthetase. FAD synthetase catalyzes the formation of flavin adenine dinucleotide from riboflavin and ATP. Thus, the cell can be further transformed to recombinantly express a gene encoding a FAD synthetase or a functional fragment thereof retaining enzymatic activity.

Since the uptake and maturation of the metalloenzymes (EgtB or OvoA) is essential for maximum efficacy, a Fe uptake system can be included in the transformed cells for producing ergothioneine. For example, the host cell can be further transformed to express a siderophore, e.g., a polynucleotide encoding a siderophore or functional fragment thereof and supplementing the medium with one or more iron salts.

It would be appreciated by one of skill in the art that the genes described herein can be obtained from a variety of sources. For example, the ovoA gene is an oxidase and comprises the nucleotide sequence with accession codes: ETA_00030 (Erwinia tasmaniensis Et1/99) or YP_001905965.

The egtD gene is a methyltransferase and comprises the nucleotide sequence with accession codes: MSMEG_6247 (Mycobacterium smegmatis str. MC2 155) or YP_890466, VERSION YP_890466.1 GI: 118473274.

The egtE gene is a C-S lyase and comprises the nucleotide sequence with accession codes: ABK70212, VERSION ABK70212.1 GI: 118169316 (pyridoxal-phosphate-dependent transferase; Mycobacterium smegmatis str. MC2 155).

The gene encoding the FAD synthetase comprises the nucleotide sequence with accession codes: D37967, VERSION D37967.1 GI: 840670 (Corynebacterium ammoniagenes gene for FAD synthetase, complete cds).

The gene encoding SAM synthetase comprises the nucleotide sequence with accession codes: K02129 REGION: 86-1240, VERSION K02129.1 GI: 146838 (E. coli metK gene coding for S-adenosylmethionine synthetase).

### Secondary Agents

In some embodiments, the method further includes contacting the cell with a second agent. Nonlimiting examples of the second agent include Nox1 inhibitor, PI3K/AKT/mTOR pathway inhibitors, and hydroxymethylglutaryl-coenzyme A (HMG-COA) reductase inhibitor. Example inhibitors are disclosed in U.S. Pat. Nos. 10,517,919, 9,656,984, and 9,072,757, the entire disclosure of which are hereby incorporated by reference. In some embodiments, the second agent may include rapamycin and/or GRN163L (or Imetelstat; by Geron).

In some embodiments, the second agent may include: idebenone, or an analog or derivative thereof; a cocoa extract; a coffee cherry extract; quinic acid, or an analog or derivative thereof; ferulic acid, or an analog or derivative thereof; a proanthocyanidin, anthocyanidin, procyanidin, or cyanidin; chlorogenic acid, or an analog or derivative thereof; a tea extract; or resveratrol or a composition derived from or chemically related to resveratrol. By way of example, the coffee cherry extract in some instances comprises one or more of chlorogenic acid, quinic acid, ferulic acid, caffeic acid or proanthocyanidins. In another example, tea extract comprises one or more polyphenols selected from EGCG (epigallocatechin-3-gallate), EGC (epigallocatechin), ECG (epicatechin-3-gallate), EC (epicatechin), GCG (gallocatechin gallate), GC (gallocatechin), C (catechin) and CG (catechin gallate). In yet another example, the composition derived from or chemically related to resveratrol is selected from the group consisting of viniferin, gnetin H, and suffruticosol B. In another example, the cocoa extract comprises a polyphenol and/or procyanidin selected from (+) catechin, (−) epicatechin, procyanidin oligomers 2 through 18, procyanidin B-5, procyanidin B-2, procyanidin A-2 and/or procyanidin C-1.

In some embodiments, the second agent may include an anti-cancer agent, such as: Abemaciclib, Abiraterone Acetate, Abraxane (Paclitaxel Albumin-stabilized Nanoparticle Formulation), ABVD, ABVE, ABVE-PC, AC, Acalabrutinib, AC-T, Actemra (Tocilizumab), Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ameluz (Aminolevulinic Acid), Amifostine, Aminolevulinic Acid, Anastrozole, Apalutamide, Aprepitant, Aranesp (Darbepoetin Alfa), Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Asparlas (Calaspargase Pegol-mknl), Atezolizumab, Avastin (Bevacizumab), Avelumab, Axicabtagene Ciloleucel, Axitinib, Azacitidine, Azedra (Iobenguane I 131), Bavencio (Avelumab), BEACOPP, Beleodaq (Belinostat), Belinostat, Bendamustine Hydrochloride, Bendeka (Bendamustine Hydrochloride), BEP, Besponsa (Inotuzumab Ozogamicin), Bevacizumab, Bexarotene, Bicalutamide, BiCNU (Carmustine), Binimetinib, Bleomycin, Blinatumomab, Blincyto (Blinatumomab), Bortezomib, Bosulif (Bosutinib), Bosutinib, Braftovi (Encorafenib), Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Calaspargase Pegol-mknl, Calquence (Acalabrutinib), Campath (Alemtuzumab), Camptosar (Irinotecan Hydrochloride), Capecitabine, CAPOX, Carac (Fluorouracil—Topical), Carboplatin, CARBOPLATIN-TAXOL, Carfilzomib, Carmustine, Carmustine Implant, Casodex (Bicalutamide), CEM, Cemiplimab-rwlc, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clofarabine, Clolar (Clofarabine), CMF, Cobimetinib, Cometriq (Cabozantinib-S-Malate), Copanlisib Hydrochloride, COPDAC, Copiktra (Duvelisib), COPP, COPP-ABV, Cosmegen (Dactinomycin), Cotellic (Cobimetinib), Crizotinib, CVP, Cyclophosphamide, Cyramza (Ramucirumab), Cytarabine, Cytarabine Liposome, Cytosar-U (Cytarabine), Dabrafenib, Dacarbazine, Dacogen (Decitabine), Dacomitinib, Dactinomycin, Daratumumab, Darbepoetin Alfa, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), Durvalumab, Duvelisib, Efudex (Fluorouracil—Topical), Eligard (Leuprolide Acetate), Elitek (Rasburicase), Ellence (Epirubicin Hydrochloride), Elotuzumab, Eloxatin (Oxaliplatin), Eltrombopag Olamine, Elzonris (Tagraxofusp-erzs), Emapalumab-lzsg, Emend (Aprepitant), Empliciti (Elotuzumab), Enasidenib Mesylate, Encorafenib, Enzalutamide, Epirubicin Hydrochloride, EPOCH, Epoetin Alfa, Epogen (Epoetin Alfa), Erbitux (Cetuximab), Eribulin Mesylate, Erivedge (Vismodegib), Erleada (Apalutamide), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Firmagon (Degarelix), Fludarabine Phosphate, Fluoroplex (Fluorouracil—Topical), Fluorouracil Injection, Fluorouracil—Topical, Flutamide, FOLFIRI, FOLFIRI-BEVACIZUMAB, FOLFIRI-CETUXIMAB, FOLFIRINOX, FOLFOX, Folotyn (Pralatrexate), Fostamatinib Disodium, FU-LV, Fulvestrant, Fusilev (Leucovorin Calcium), Gamifant (Emapalumab-lzsg), Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gilteritinib Fumarate, Gleevec (Imatinib Mesylate), Gliadel Wafer (Carmustine Implant), Glucarpidase, Goserelin Acetate, Granisetron, Granisetron Hydrochloride, Granix (Filgrastim), Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idarubicin Hydrochloride, Idelalisib, Idhifa (Enasidenib Mesylate), Ifex (Ifosfamide), Ifosfamide, IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iobenguane I 131, Ipilimumab, Iressa (Gefitinib), Irinotecan Hydrochloride, Irinotecan Hydrochloride Liposome, Istodax (Romidepsin), Ivosidenib, Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Larotrectinib Sulfate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Levulan Kerastik (Aminolevulinic Acid), Libtayo (Cemiplimab-rwlc), LipoDox (Doxorubicin Hydrochloride Liposome), Lomustine, Lonsurf (Trifluridine and Tipiracil Hydrochloride), Lorbrena (Lorlatinib), Lorlatinib, Lumoxiti (Moxetumomab Pasudotox-tdfk), Lupron (Leuprolide Acetate), Lupron Depot (Leuprolide Acetate), Lutathera (Lutetium Lu 177-Dotatate), Lutetium (Lu 177-Dotatate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Mektovi (Binimetinib), Melphalan, Melphalan Hydrochloride, Mercaptopurine, Mesna, Mesnex (Mesna), Methotrexate, Methylnaltrexone Bromide, Midostaurin, Mitomycin C, Mitoxantrone Hydrochloride, Mogamulizumab-kpkc, Moxetumomab Pasudotox-tdfk, Mozobil ((Mechlorethamine Hydrochloride), MVAC, Myleran (Busulfan), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Plerixafor, Pomalidomide, Pomalyst (Pomalidomide), Ponatinib Hydrochloride, Portrazza (Necitumumab), Poteligeo (Mogamulizumab-kpkc), Pralatrexate, Prednisone, Procarbazine Hydrochloride, Procrit (Epoetin Alfa), Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, Ravulizumab-cwvz, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Retacrit (Epoetin Alfa), Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sancuso (Granisetron), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sustol (Granisetron), Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagraxofusp-erzs, Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Tavalisse (Fostamatinib Disodium), Taxol (Paclitaxel), Taxotere (Docetaxel), Tecentriq (Atezolizumab), Temodar (Temozolomide), Temozolomide, Temsirolimus, Thalidomide, Thalomid (Thalidomide), Thioguanine, Thiotepa, Tibsovo (Ivosidenib), Tisagenlecleucel, Tocilizumab, Tolak (Fluorouracil—Topical), Topotecan Hydrochloride, Toremifene, Torisel (Temsirolimus), Totect (Dexrazoxane Hydrochloride), TPF, Trabectedin, Trametinib, Trastuzumab, Treanda (Bendamustine Hydrochloride), Trexall (Methotrexate), Trifluridine and Tipiracil Hydrochloride, Trisenox (Arsenic Trioxide), Tykerb (Lapatinib Ditosylate), Ultomiris (Ravulizumab-cwvz), Unituxin (Dinutuximab), Uridine Triacetate, VAC, Valrubicin, Valstar (Valrubicin), Vandetanib, VAMP, Varubi (Rolapitant Hydrochloride), Vectibix (Panitumumab), VeIP, Velcade (Bortezomib), Vemurafenib, Venclexta (Venetoclax), Venetoclax, Verzenio (Abemaciclib), Vidaza (Azacitidine), Vinblastine Sulfate, Vincristine Sulfate, Vincristine Sulfate Liposome, Vinorelbine Tartrate, VIP, Vismodegib, Vistogard (Uridine Triacetate), Vitrakvi (Larotrectinib Sulfate), Vizimpro (Dacomitinib), Voraxaze (Glucarpidase), Vorinostat, Votrient (Pazopanib Hydrochloride), Vyxeos (Daunorubicin Hydrochloride and Cytarabine Liposome), Xalkori (Crizotinib), Xeloda (Capecitabine), XELIRI, XELOX, Xgeva (Denosumab), Xofigo (Radium 223 Dichloride), Xospata (Gilteritinib Fumarate), Xtandi (Enzalutamide), Yervoy (Ipilimumab), Yescarta (Axicabtagene Ciloleucel), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), Zytiga (Abiraterone Acetate).

In some embodiments, the second agent may include treatments for Alzheimer's, including donepezil, Aricept, galantamine, Razadyne, memantine, Namenda, rivastigmine, and Exelon.

### Compositions for Extending Lifespan

Also within the scope of this disclosure are the pharmaceutical compositions for modulating the lifespan of a cell, tissue, organ or organism. The pharmaceutical compositions may include ergothioneine, or an analog, a derivative, or a pharmaceutically acceptable prodrug, a pharmaceutically active metabolite, a pharmaceutically acceptable salt thereof.

Pharmaceutical compositions for use in accordance with the present methods may be formulated in a conventional manner using one or more physiologically acceptable carriers or excipients. Thus, ergothioneine and its analogs/variants described herein that modulate the protein or expression level of nucleic acids described herein, and their physiologically acceptable salts and solvates may be formulated for administration by, for example, injection, inhalation or insufflation (either through the mouth or the nose) or oral, buccal, parenteral or rectal administration. In one embodiment, the agent is administered locally, e.g., at the site where the target cells are present, such as by the use of a patch.

Pharmaceutical compositions can be formulated for a variety of loads of administration, including systemic and topical or localized administration. Techniques and formulations generally may be found in Remmington's Pharmaceutical Sciences, Meade Publishing Co., Easton, PA. For systemic administration, injection is preferred, including intramuscular, intravenous, intraperitoneal, and subcutaneous. For injection, the agents can be formulated in liquid solutions, preferably in physiologically compatible buffers such as Hank's solution or Ringer's solution. In addition, the agents may be formulated in solid form and redissolved or suspended immediately prior to use. Lyophilized forms are also included.

For oral administration, the pharmaceutical compositions may take the form of, for example, tablets, lozenges, or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicles before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated to give controlled release of the active compound.

Pharmaceutical compositions that may oxidize and lose biological activity, especially in a liquid or semisolid form, may be prepared in a nitrogen atmosphere or sealed in a type of capsule and/or foil package that excludes oxygen (e.g., Capsugel™).

For administration by inhalation, the agents may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, e.g., gelatin, for use in an inhaler or insufflator may be formulated containing a powder mix of the agent and a suitable powder base such as lactose or starch.

Pharmaceutical compositions may be formulated for parenteral administration by injection, e.g., by bolus injection or continuous infusion. Formulations for injection may be presented in unit dosage form, e.g., in ampoules or in multi-dose containers, with an added preservative. The agents may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use. The agents may also be formulated in rectal compositions such as suppositories or retention enemas, e.g., containing conventional suppository bases such as cocoa butter or other glycerides.

In addition to the formulations described previously, pharmaceutical compositions may also be formulated as a depot preparation. Such long-acting formulations may be administered by implantation (for example subcutaneously or intramuscularly) or by intramuscular injection. Thus, for example, the agents may be formulated with suitable polymeric or hydrophobic materials (for example as an emulsion in an acceptable oil) or ion exchange resins, or as sparingly soluble derivatives, for example, as a sparingly soluble salt. Controlled release formula also includes patches, e.g., transdermal patches. Patches may be used with a sonic applicator that deploys ultrasound in a unique combination of waveforms to introduce drug molecules through the skin that normally could not be effectively delivered transdermally.

Pharmaceutical compositions (including cosmetic preparations) may comprise from about 0.00001 to 100% such as from 0.001 to 10% or from 0.1% to 5% by weight of one or more agents described herein.

A pharmaceutical composition described herein can also be incorporated into a topical formulation containing a topical earner that is generally suited to topical drug administration and comprising any such material known in the art. The topical carrier may be selected so as to provide the composition in the desired form, e.g., as an ointment, lotion, cream, microemulsion, gel, oil, solution, or the like, and may be comprised of a material of either naturally occurring or synthetic origin. It is preferable that the selected carrier not adversely affect the active agent or other components of the topical formulation. Examples of suitable topical carriers for use herein include water, alcohols and other nontoxic organic solvents, glycerin, mineral oil, silicone, petroleum jelly, lanolin, fatty acids, vegetable oils, parabens, waxes, and the like.

Formulations may be colorless, odorless ointments, lotions, creams, microemulsions, and gels. Pharmaceutical compositions may be incorporated into ointments, which generally are semisolid preparations which are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency or the like. As with other carriers or vehicles, an ointment base should be inert, stable, nonirritating and nonsensitizing. As explained in Remington's, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin, and hydrophilic petrolatum. Emulsion ointment bases are either water-in-oil (W/O) emulsions or oil-in-water (O/W) emulsions, and include, for example, cetyl alcohol, glyceryl monostearate, lanolin, and stearic acid. Exemplary water-soluble ointment bases are prepared from polyethylene glycols (PEGs) of varying molecular weight; again, reference may be had to Remington's, supra, for further information.

Pharmaceutical compositions may be incorporated into lotions, which generally are preparations to be applied to the skin surface without friction, and are typically liquid or semiliquid preparations in which solid particles, including the active agent, are present in a water or alcohol base. Lotions are usually suspensions of solids, and may comprise a liquid oily emulsion of the oil-in-water type. Lotions are preferred formulations for treating large body areas, because of the ease of applying a more fluid composition. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethylcellulose, or the like. An exemplary lotion formulation for use in conjunction with the present method contains propylene glycol mixed with hydrophilic petrolatum such as that which may be obtained under the trademark Aquaphor™ from Beiersdorf, Inc. (Norwalk, Conn.).

Pharmaceutical compositions may be incorporated into creams, which generally are viscous liquid or semisolid emulsions, either oil-in-water or water-in-oil. Cream bases are water-washable and contain an oil phase, an emulsifier and an aqueous phase. The oil phase is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation, as explained in Remington's, supra, is generally a nonionic, anionic, cationic or amphoteric surfactant.

Pharmaceutical compositions may be incorporated into microemulsions, which generally are thermodynamically stable, isotropically clear dispersions of two immiscible liquids, such as oil and water, stabilized by an interfacial film of surfactant molecules (Encyclopedia of Pharmaceutical Technology (New York: Marcel Dekker, 1992), volume 9). For the preparation of microemulsions, surfactant (emulsifier), co-surfactant (co-emulsifier), an oil phase and a water phase are necessary. Suitable surfactants include any surfactants that are useful in the preparation of emulsions, e.g., emulsifiers that are typically used in the preparation of creams. The co-surfactant (or “co-emulsifier”) is generally selected from the group of polyglycerol derivatives, glycerol derivatives, and fatty alcohols. Preferred emulsifier/co-emulsifier combinations are generally although not necessarily selected from the group consisting of: glyceryl monostearate and polyoxyethylene stearate; polyethylene glycol and ethylene glycol palmitostearate; and caprylic and capric triglycerides and oleoyl macrogolglycerides. The water phase includes not only water but also, typically, buffers, glucose, propylene glycol, polyethylene glycols, preferably lower molecular weight polyethylene glycols (e.g., PEG 300 and PEG 400), and/or glycerol, and the like, while the oil phase will generally comprise, for example, fatty acid esters, modified vegetable oils, silicone oils, mixtures of mono-di-and triglycerides, mono-and di-esters of PEG (e.g., oleoyl macrogol glycerides), etc.

Pharmaceutical compositions may be incorporated into gel formulations, which generally are semisolid systems consisting of either suspension made up of small inorganic particles (two-phase systems) or large organic molecules distributed substantially uniformly throughout a carrier liquid (single phase gels). Single phase gels can be made, for example, by combining the active agent, a carrier liquid and a suitable gelling agent such as tragacanth (at 2 to 5%), sodium alginate (at 2-10%), gelatin (at 2-15%), methylcellulose (at 3-5%), sodium carboxymethylcellulose (at 2-5%), carbomer (at 0.3-5%) or polyvinyl alcohol (at 10-20%) together and mixing until a characteristic semisolid product is produced. Other suitable gelling agents include methylhydroxycellulose, polyoxyethylene-polyoxypropylene, hydroxyethylcellulose, and gelatin. Although gels commonly employ aqueous carrier liquid, alcohols and oils can be used as the carrier liquid as well.

Various additives, known to those skilled in the art, may be included in formulations, e.g., topical formulations. Examples of additives include, but are not limited to, solubilizers, skin permeation enhancers, opacifiers, preservatives (e.g., antioxidants), gelling agents, buffering agents, surfactants (particularly nonionic and amphoteric surfactants), emulsifiers, emollients, thickening agents, stabilizers, humectants, colorants, fragrance, and the like. Inclusion of solubilizers and/or skin permeation enhancers is particularly preferred, along with emulsifiers, emollients, and preservatives. An optimum topical formulation comprises approximately: 2 wt. % to 60 wt. %, preferably 2 wt. % to 50 wt. %, solubilizer and/or skin permeation enhancer; 2 wt. % to 50 wt. %, preferably 2 wt. % to 20 wt. %, emulsifiers; 2 wt. % to 20 wt. % emollient; and 0.01 to 0.2 wt. % preservative, with the active agent and carrier (e.g., water) making of the remainder of the formulation. A skin permeation enhancer serves to facilitate passage of therapeutic levels of active agent to pass through a reasonably sized area of unbroken skin. Suitable enhancers are well known in the art and include, for example: lower alkanols such as methanol ethanol and 2-propanol; alkyl methyl sulfoxides such as dimethylsulfoxide (DMSO), decylmethylsulfoxide (C.sub.10 MSO) and tetradecylmethyl sulfoxide; pyrrolidones such as 2-pyrrolidone, N-methyl-2-pyrrolidone and N-(-hydroxyethyl) pyrrolidone; urea; N,N-diethyl-m-toluamide; C.sub.2-C. sub.6 alkane diols; miscellaneous solvents such as dimethylformamide (DMF), N,N-dimethylacetamide (DMA) and tetrahydrofurfuryl alcohol; and the 1-substituted azacycloheptan-2-ones, particularly 1-n-dodecylcyclazacycloheptan-2-one (laurocapram; available under the trademark AzoneRTM from Whitby Research Incorporated, Richmond, Va.).

Examples of solubilizers include, but are not limited to, the following: hydrophilic ethers such as diethylene glycol monoethyl ether (ethoxydiglycol, available commercially as Transcutol™) and diethylene glycol monoethyl ether oleate (available commercially as Softcutol™); polyethylene castor oil derivatives such as polyoxy 35 castor oil, polyoxy 40 hydrogenated castor oil, etc.; polyethylene glycol, particularly lower molecular weight polyethylene glycols such as PEG 300 and PEG 400, and polyethylene glycol derivatives such as PEG-8 caprylic/capric glycerides (available commercially as Labrasol™); alkyl methyl sulfoxides such as DMSO; pyrrolidones such as 2-pyrrolidone and N-methyl-2-pyrrolidone; and DMA. Many solubilizers can also act as absorption enhancers. A single solubilizer may be incorporated into the formulation, or a mixture of solubilizers may be incorporated therein.

Suitable emulsifiers and co-emulsifiers include, without limitation, those emulsifiers and co-emulsifiers described with respect to microemulsion formulations. Emollients include, for example, propylene glycol, glycerol, isopropyl myristate, polypropylene glycol-2 (PPG-2) myristyl ether propionate, and the like.

Other active agents may also be included in formulations, e.g., anti-inflammatory agents, analgesics, antimicrobial agents, antifungal agents, antibiotics, vitamins, antioxidants, and sunblock agents commonly found in sunscreen formulations including, but not limited to, anthranilates, benzophenones (particularly benzophenone-3), camphor derivatives, cinnamates (e.g., octyl methoxycinnamate), dibenzoyl methanes (e.g., butyl methoxydibenzoyl methane), p-aminobenzoic acid (PABA) and derivatives thereof, and salicylates (e.g., octyl salicylate). In certain topical formulations, the active agent is present in an amount in the range of approximately 0.25 wt. % to 75 wt. % of the formulation, preferably in the range of approximately 0.25 wt. % to 30 wt. % of the formulation, more preferably in the range of approximately 0.5 wt. % to 15 wt. % of the formulation, and most preferably in the range of approximately 1.0 wt. % to 10 wt. % of the formulation. Topical skin treatment compositions can be packaged in a suitable container to suit its viscosity and intended use by the consumer. For example, a lotion or cream can be packaged in a bottle or a roll-ball applicator, or a propellant-driven aerosol device or a container fitted with a pump suitable for finger operation. When the composition is a cream, it can simply be stored in a non-deformable bottle or squeeze container, such as a tube or a lidded jar. The composition may also be included in capsules such as those described in U.S. Pat. No. 5,063,507. Accordingly, also provided are closed containers containing a cosmetically acceptable composition.

In some embodiments, a pharmaceutical formulation is provided for oral or parenteral administration, in which case the formulation may comprise an activating compound-containing microemulsion as described above, and may contain alternative pharmaceutically acceptable carriers, vehicles, additives, etc. particularly suited to oral or parenteral drug administration. Alternatively, an activating compound-containing microemulsion may be administered orally or parenterally substantially as described above, without modification.

Dosages for a particular individual can be determined by one of ordinary skill in the art using conventional considerations, (e.g., by means of an appropriate, conventional pharmacological protocol). A physician may, for example, prescribe a relatively low dose at first, subsequently increasing the dose until an appropriate response is obtained. The dose administered to an individual is sufficient to effect a beneficial therapeutic response in the individual over time, or, e.g., to reduce symptoms, or other appropriate activity, depending on the application. The dose is determined by the efficacy of the particular formulation, and the activity, stability or serum half-life of the miRNA employed and the condition of the individual, as well as the body weight or surface area of the individual to be treated. The size of the dose is also determined by the existence, nature, and extent of any adverse side-effects that accompany the administration of a particular vector, formulation, or the like in a particular individual.

Also provided herein are kits, e.g., kits for therapeutic purposes, including kits for modulating ageing, apoptosis, and for treating diseases, e.g., those described herein. A kit may comprise one or more agents such as include ergothioneine, or an analog, a derivative, or a pharmaceutically acceptable prodrug, a pharmaceutically active metabolite, a pharmaceutically acceptable salt thereof described herein, a second agent as described above and optionally devices for contacting cells with the agents. Devices include syringes, stents and other devices for introducing an agent into a subject or applying it to the skin of a subject. Further, a kit may also contain components for measuring a factor, e.g., described above, such as a protein or transcript level, e.g., in tissue samples. The kits may also include instructions for use.

A kit may include an agent for measuring the activity and or expression level of DAF-16, FOXO1, FOXO2, FOXO3, FOXO6, RAB-1, PMK-1, AKT-1, AKT2, JNK-1, PDK-1, or DAF-2 or other genes in the daf-16 signaling pathway.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2^{nd}Ed., ed. By Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Patent No: 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N. Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N. Y., 1986).

### Additional Definitions

To aid in understanding the detailed description of the compositions and methods a, ccording to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

As used herein, the terms “subject” and “patient” are used interchangeably irrespective of whether the subject has or is currently undergoing any form of treatment. As used herein, the terms “subject” and “subjects” may refer to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous monkey, chimpanzee, etc.) and a human). The subject may be a human or a non-human. In this context, a “normal,” “control,” or “reference” subject, patient or population is/are one(s) that exhibit(s) no detectable disease or disorder, respectively.

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent” which is a biologically, physiologically, or pharmacologically active substance (or substances) that acts locally or systemically in a subject.

The term “therapeutic agent” is art-recognized and refers to any chemical moiety that is a biologically, physiologically, or pharmacologically active substance that acts locally or systemically in a subject. The term also means any substance intended for use in the diagnosis, cure, mitigation, treatment or prevention of disease or in the enhancement of desirable physical or mental development and/or conditions in an animal or human.

The term “therapeutic effect” is art-recognized and refers to a local or systemic effect in animals, particularly mammals, and more particularly humans caused by a pharmacologically active substance. The phrase “therapeutically-effective amount” means that amount of such a substance that produces some desired local or systemic effect at a reasonable benefit/risk ratio applicable to any treatment. The therapeutically effective amount of such substance will vary depending upon the subject and disease or condition being treated, the weight and age of the subject, the severity of the disease or condition, the manner of administration and the like, which can readily be determined by one of ordinary skill in the art. For example, certain compositions described herein may be administered in a sufficient amount to produce a desired effect at a reasonable benefit/risk ratio applicable to such treatment.

The term “derivative” as used herein refers to a chemical substance related structurally to another, i.e., an “original” substance, which can be referred to as a “parent” compound. A “derivative” can be made from the structurally-related parent compound in one or more steps. The phrase “closely related derivative” means a derivative whose molecular weight does not exceed the weight of the parent compound by more than 50%. The general physical and chemical properties of a closely related derivative are also similar to the parent compound.

“Apoptosis” refers to the process by which cells are programmed to die or lose viability. Commonly triggered by cytochrome leakage from the mitochondria and accompanied by signaling cascades (caspases and other proteins) resulting in decreased mitochondrial and energy potential via the electron transport system, an buildup of reactive oxygen species and free radical and loss of membrane integrity.

The term “age-related disease/disorder/condition” refers to disorders associated with senescence. Representative age-related disorders include, but are not limited to, progeria, wrinkles/skin blemishes/liver spots, obesity, locomotor dysfunction, sterility.

The term “orthologues” refers to separate occurrences of the same gene in multiple species. The separate occurrences have similar, albeit nonidentical, amino acid sequences, the degree of sequence similarity depending, in part, upon the evolutionary distance of the species from a common ancestor having the same gene.

As used herein, the term “paralogues” indicates separate occurrences of a gene in one species. The separate occurrences have similar, albeit nonidentical, amino acid sequences, the degree of sequence similarity depending, in part, upon the evolutionary distance from the gene duplication event giving rise to the separate occurrences.

As used herein, the term “homologs” is generic to “orthologues” and “paralogues.”

“Healthy longevity” refers to the concept of having entire organisms (as well as organs, tissues, and individual cells) at optimal genetic and functional health. While not limited to these issues, this means for example that the DNA is not significantly damaged or mutated and is in a state comparable to the configuration that would occur in a natural healthy infant or fetus. In other embodiments, the DNA has been altered to be equivalent or better than that status through, e.g., repair or genetic engineering. Similarly, in some embodiments, the mitochondrial number and/or function and/or respiratory efficiency are similarly optimal or supra-optimal. Metabolic pathways and immune function also may be likewise optimized, and existing environmental damage may have been repaired. Intrinsic chronologic ageing and/or oxidative stress damage from normal cellular processes such as free radical damage within mitochondria have also been mitigated or reversed or repaired or otherwise restored to a youthful optimally functional status or a close approximation of the same. Unhealthy cells, including even cancerous cells, which have not been repaired, are eliminated via apoptosis or the death of these cells has been modulated to be accelerated. Significantly gene expression patterns and pathways have been reregulated, or reset or resignalled in such a fashion as to optimize the function and health of the cells and by extension the tissues, organs, and organisms that these cells comprise. One end result of at least one or perhaps more of these processes is that the cells achieve maximal longevity or lifespan and/or function optimally or at maximal efficiency and effectiveness for the duration of their lifespan. Understanding that such a process may not be undertaken until substantial damage from ageing, disease, diet, injury, environmental exposure, medication or medical therapy side effects, etc. it is understood that even a partial achievement of one or more of these goals would improve the length of the lifespan or make the remaining lifespan duration healthier. Modulating cell function to achieve one or more of these goals is then a means of producing a state termed healthy longevity. The modulation of cell activity to accomplish this may involve in some instances modulating to kill cells prematurely and in a manner diminish the cells health to the point of cell death in order to remove unhealthy cells which may harm the tissue, organ or organism or even which may stimulate the creation and replacement of the unhealthy or sub-optimally healthy cell(s) with new cells via cell division of healthy cells, biogenesis of new cells or replacement of cells via stem cells or autologous transplant or allograft or other types of transplanted cells including genetically engineered cells for transplantation. The treatment of such cells with the process of this invention prior to or after transplantation is also envisioned as a means to produce healthy longevity in these ‘new’ cells.

“Sample,” “test sample,” and “patient sample” may be used interchangeably herein. The sample can be a sample of, serum, urine plasma, amniotic fluid, cerebrospinal fluid, cells (e.g., antibody-producing cells) or tissue. Such a sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art. The terms “sample” and “biological sample” as used herein generally refer to a biological material being tested for and/or suspected of containing an analyte of interest such as antibodies. The sample may be any tissue sample from the subject. The sample may comprise protein from the subject.

Any cell type, tissue, or bodily fluid may be utilized to obtain a sample. Such cell types, tissues, and fluid may include sections of tissues such as biopsy and autopsy samples, frozen sections taken for histologic purposes, blood (such as whole blood), plasma, serum, sputum, stool, tears, mucus, saliva, hair, skin, red blood cells, platelets, interstitial fluid, ocular lens fluid, cerebral spinal fluid, sweat, nasal fluid, synovial fluid, menses, amniotic fluid, semen, etc. Cell types and tissues may also include lymph fluid, ascetic fluid, gynecological fluid, urine, peritoneal fluid, cerebrospinal fluid, a fluid collected by vaginal rinsing, or a fluid collected by vaginal flushing. A tissue or cell type may be provided by removing a sample of cells from an animal, but can also be accomplished by using previously isolated cells (e.g., isolated by another person, at another time, and/or for another purpose). Archival tissues, such as those having treatment or outcome history, may also be used. Protein purification may not be necessary.

Methods well known in the art for collecting, handling and processing urine, blood, serum, and plasma, and other body fluids, can be used in the practice of the present disclosure, for instance, when the antibodies provided herein are employed as immunodiagnostic reagents, and/or in an immunoassay kit. The test sample can comprise further moieties in addition to the analyte of interest, such as antibodies, antigens, haptens, hormones, drugs, enzymes, receptors, proteins, peptides, polypeptides, oligonucleotides or polynucleotides. For example, the sample can be a whole blood sample obtained from a subject. It can be necessary or desired that a test sample, particularly whole blood, be treated prior to immunoassay as described herein, e.g., with a pretreatment reagent. Even in cases where pretreatment is not necessary, pretreatment optionally can be done for mere convenience (e.g., as part of a regimen on a commercial platform). The sample may be used directly as obtained from the subject or following a pretreatment to modify a characteristic of the sample. Pretreatment may include extraction, concentration, inactivation of interfering components, and/or the addition of reagents.

The term “allele” as used herein refers to one or more alternative forms of a gene occupying a given locus on a chromosome.

The terms “polypeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, by disulfide bond formation, glycosylation, lipidation, acetylation, phosphorylation, or any other manipulation, such as conjugation with a labeling component. As used herein the term “amino” acid” includes natural and/or unnatural or synthetic amino acids, including glycine and both the D or L optical isomers, and amino acid analogs and peptidomimetics.

The term “amino acid sequence” refers to an amino acid sequence of a protein molecule, “amino acid sequence” and like terms, such as “polypeptide” or “protein” are not meant to limit the amino acid sequence to the complete, native amino acid sequence associated with the recited protein molecule. Furthermore, an “amino acid sequence” can be deduced from the nucleic acid sequence encoding the protein.

The term “homolog” or “homologous” when used in reference to a polypeptide refers to a high degree of sequence identity between two polypeptides, or to a high degree of similarity between the three-dimensional structure or to a high degree of similarity between the active site and the mechanism of action. In a preferred embodiment, a homolog has a greater than 60% sequence identity, and more preferably greater than 75% sequence identity, and still more preferably greater than 90% sequence identity, with a reference sequence. The term “substantial identity,” as applied to polypeptides, means that two peptide sequences, when optimally aligned, such as by the programs GAP or BESTFIT using default gap weights, share at least 75% sequence identity.

The term “gene” refers to a nucleic acid (e.g., DNA or RNA) sequence that comprises coding sequences necessary for the production of an RNA, or a polypeptide or its precursor (e.g., proinsulin). A functional polypeptide can be encoded by a full-length coding sequence or by any portion of the coding sequence as long as the desired activity or functional properties (e.g., enzymatic activity, ligand binding, signal transduction, etc.) of the polypeptide are retained. The term “portion” when used in reference to a gene refers to fragments of that gene. The fragments may range in size from a few nucleotides to the entire gene sequence minus one nucleotide. Thus, “a nucleotide comprising at least a portion of a gene” may comprise fragments of the gene or the entire gene.

The term “gene” also encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns, therefore, are absent in the messenger RNA (mRNA) transcript. The mRNA functions during translation to specify the sequence or order of amino acids in a nascent polypeptide.

The term “recombinant” when made in reference to a nucleic acid molecule refers to a nucleic acid molecule which is comprised of segments of nucleic acid joined together by means of molecular biological techniques. The term “recombinant,” when made in reference to a protein or a polypeptide, refers to a protein molecule which is expressed using a recombinant nucleic acid molecule.

As used herein, “expression” refers to the process by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism such as a non-human animal.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

As used herein, the term “administering” refers to the delivery of cells by any route including, without limitation, oral, intranasal, intraocular, intravenous, intraosseous, intraperitoneal, intraspinal, intramuscular, intra-articular, intraventricular, intracranial, intralesional, intratracheal, intrathecal, subcutaneous, intradermal, transdermal, or transmucosal administration.

It is noted here that, as used in this specification and the appended claims, the singular forms “a,” “an,” and “the” include plural reference unless the context clearly dictates otherwise. The terms “including,” “comprising,” “containing,” or “having” and variations thereof are meant to encompass the items listed thereafter and equivalents thereof as well as additional subject matter unless otherwise noted.

The phrases “in one embodiment,” “in various embodiments,” “in some embodiments,” and the like are used repeatedly. Such phrases do not necessarily refer to the same embodiment, but they may unless the context dictates otherwise.

The terms “and/or” or “/” means any one of the items, any combination of the items, or all of the items with which this term is associated.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

As used herein, the term “each,” when used in reference to a collection of items, is intended to identify an individual item in the collection but does not necessarily refer to every item in the collection. Exceptions can occur if explicit disclosure or context clearly dictates otherwise.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise.

In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present invention. Nothing herein is to be construed as an admission that the present invention is not entitled to antedate such publication by virtue of prior invention. Further, the dates of publication provided may be different from the actual publication dates which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### IV. EXAMPLES

### Example 1

In this example, the biological functions of ergothioneine were investigated using (C. elegans as the model system. Using (C. elegans as the model has several prominent advantages, including short life cycle, simple experimental manipulation, and rich collection of publicly available genetic mutants (e.g., Caenorhabditis Genetics Center). In addition, its genome comprises 2/3 of human disease-related genes.

To test the biological effect of ergothioneine, the first set of experiments was to test the toxicity level of ergothioneine on the worm model. In humans, ergothioneine is enriched up to 2 mM concentration in many parts of the body. Surprisingly, even at concentrations as low as 50 μM, ergothioneine demonstrated a robust life-extension effect on N2 C. elegans (FIG. 2A).

Encouraged by this result, ergothioneine concentration dependence was then examined. At 150 μM, the lifespan of the wild type worm can be extended by 30-40% (FIG. 2A). Over the last two decades, there is numerous evidence supporting the life-extension effect by caloric restriction from various model systems (yeast, worm, fly, and mouse). Thus, the effect of ergothioneine on worm Eat-2 mutant was also examined. Mutations in eat genes resulted in partial starvation of the worm by disrupting the function of the pharynx and significantly increase the lifespan. Interestingly, ergothioneine can further extend the life of Eat-2 worm mutant (FIG. 2B). This result suggests that ergothioneine can work synergistically with the currently known caloric restriction approach to further extend the lifespan (FIG. 2C).

### Example 2

Encouraged by the clear life-extension effect on worm model under regular culture conditions in the above example, the protective effect of ergothioneine under stresses was examined. In the first experiment, the worm was treated with paraquat to induce oxidative stress. Under the paraquat-induced oxidative stress conditions, N2 worms' life span was decreased by nearly 50% (FIG. 2C vs. FIG. 2A). As anticipated, when ergothioneine was added to worms under oxidative stress conditions, it clearly offers protective effects and alleviates the detrimental effects of oxidative stress (FIG. 2C).

Besides the oxidative stress, the protective effect of ergothioneine for the worm under heat shock conditions was also examined. After the worms were cultured multiple days, they were then moved from 20° C. to 42° C. Under such a heat-shock condition, 50% of the worm died within 3 hours. However, in the presence of a few hundred micromolar concentration of ergothioneine, it extends N2 worm's average survival time from 3 hours to nearly 8-9 hours (FIG. 2D). Therefore, besides its life-extension effects under regular culture conditions, ergothioneine clearly offers protective effects to worms under oxidative stress and heat shock conditions.

### Example 4

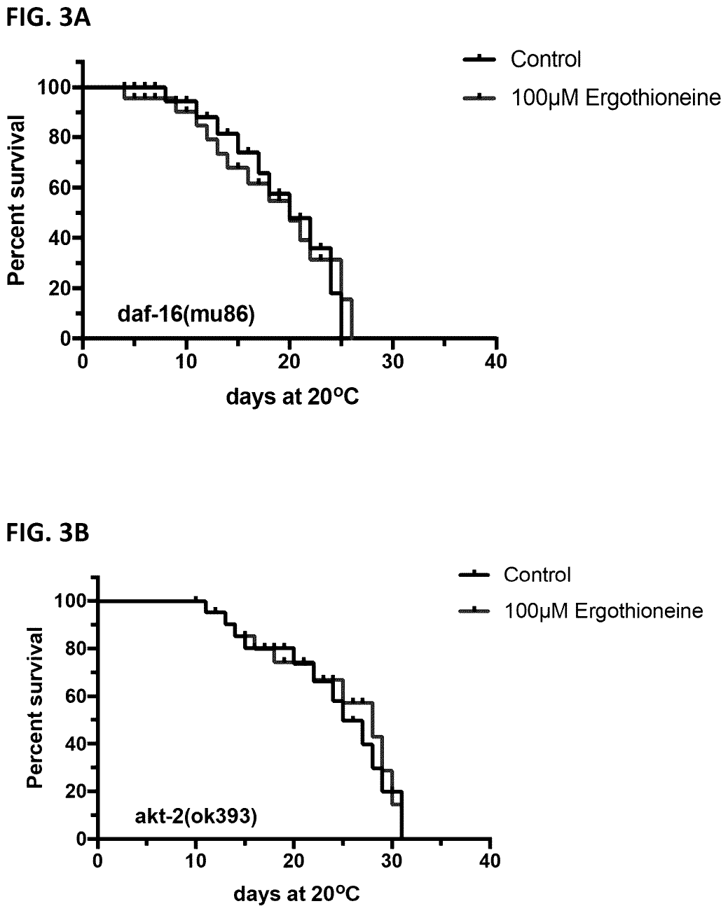

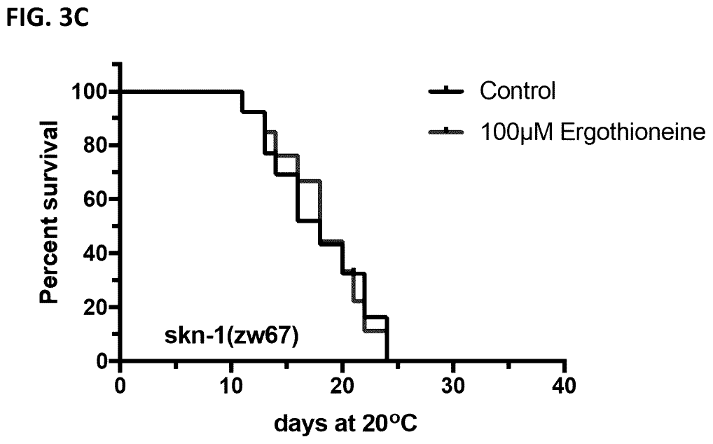

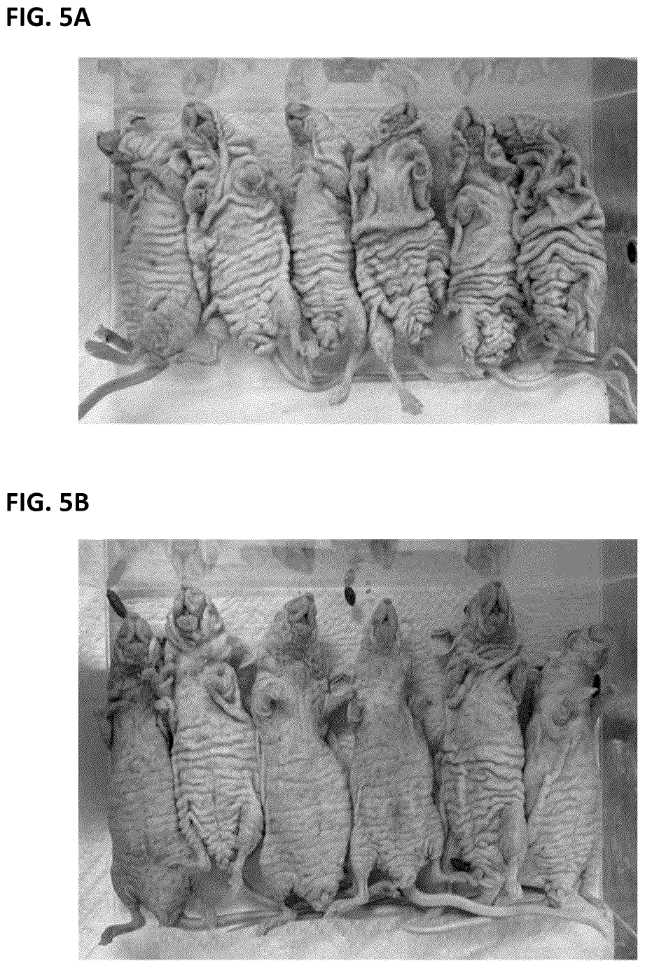

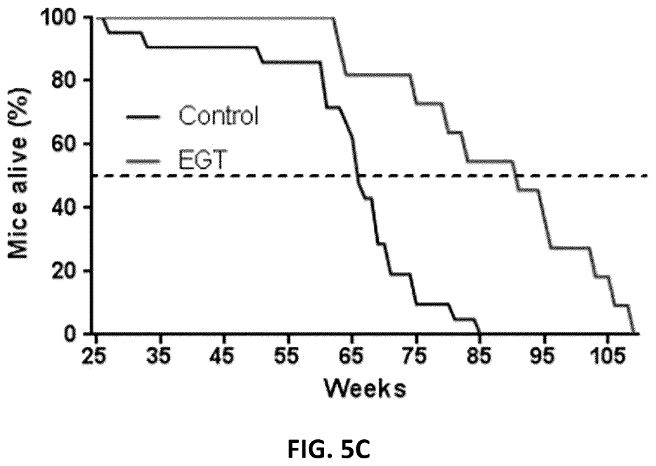

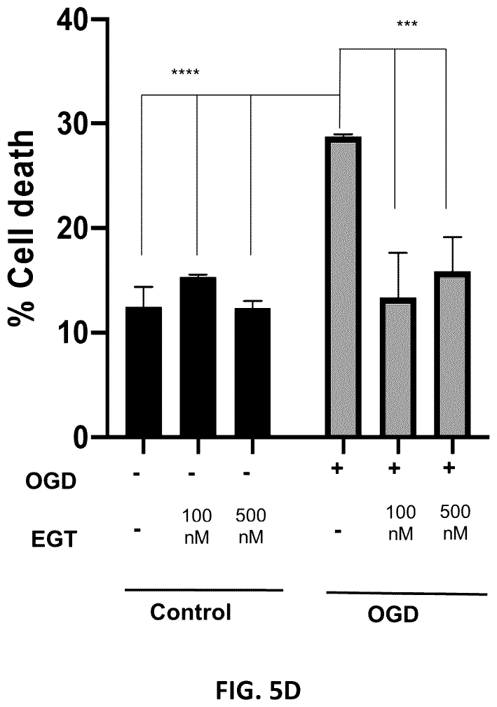

After demonstrating the life-extension effect of ergothioneine under both regular and stress conditions on the N2 worm model, it remained to be determined what signaling pathway might be involved. To address this issue, the large collection of C. elegans mutants in Caenorhabditis Genetics Center was again utilized in this example. Among the mutants examined, the daf-16 mutant results stood out (FIGS. 3A, 3B, and 3C). Insulin/IGF-1 signaling (IIS) pathway has been demonstrated to be related to worm life-span. Among genes of the IIS pathway, daf-2 encodes an insulin-like receptor, and it is the starting point of the pathway that eventually leads to daf-16. Daf-16 is the hub, where information from several different pathways merge and then use to regulate many physiological processes. Worm daf-16 mutant has an average life-span that is almost twice as long as the N2 worm (FIGS. 3A, 3B, and 3C; FIGS. 4-6). Interestingly, when ergothioneine was added onto the daf-16 mutant worm, the life-extension effect was abolished. After decades of searching for ergothioneine related working mechanism, for the first time, this result now clearly shows that ergothioneine most likely work through daf-16 related signaling pathways. FIG. 5C shows that ergothioneine extends the median lifespan by at least 40% and maximum lifespan by more than at least 30%, as compared to the mice that have not been treated with ergothioneine. FIG. 5D shows that ergothioneine reverses cell death cause by oxygen glucose deprivation for primary cortical neuron culture.

### Example 5

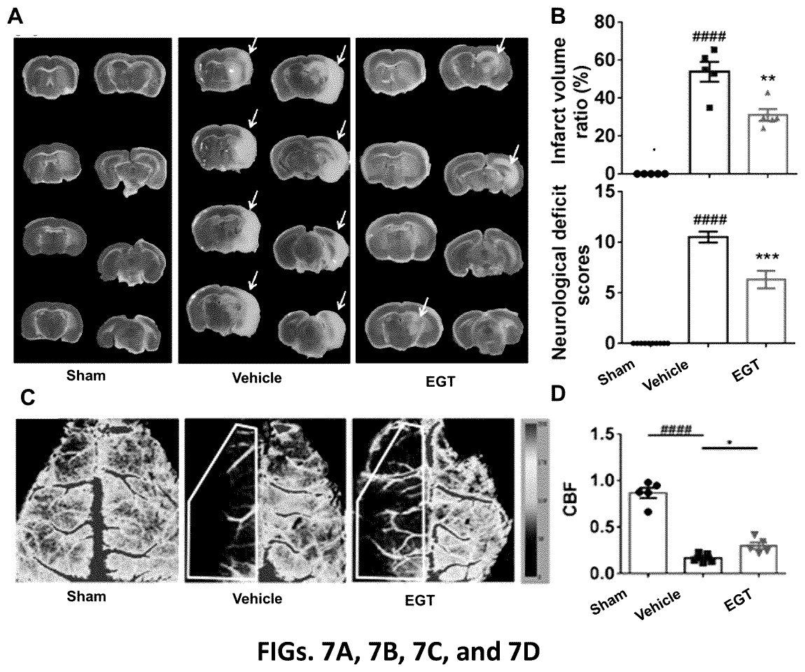

FIG. 7 shows protective effects of ergothioneine on a stroke middle cerebral artery occlusion (MCAO) model. The MCAO model is a stroke model that uses mice and rats. The model involves inserting a surgical filament into the external carotid artery and threading it into the internal carotid artery. The filament is inserted to block blood flow for a set period of time, usually 30-120 minutes. The filament is then removed to allow reperfusion, resulting in an ischemic stroke. A sham mouse is a control animal used in partial bladder outlet obstruction experiments. Sham procedures are performed on a control group participant to ensure they experience the same effects as those who receive a true operation.

FIG. 7B shows infarct volume of Sham, vehicle, and ergothioneine treated mice. As shown, ergothioneine reduces the infarct size by at least 50%. FIG. 7C shows neurological deficit Sham, vehicle, and ergothioneine treated mice. As shown, ergothioneine reduces neurological deficit by at least 40%. FIG. 7D shows cerebral blood flow (CBF) of Sham, vehicle, and ergothioneine treated mice. As shown, ergothioneine reduces cerebral blood flow by at least 60%.

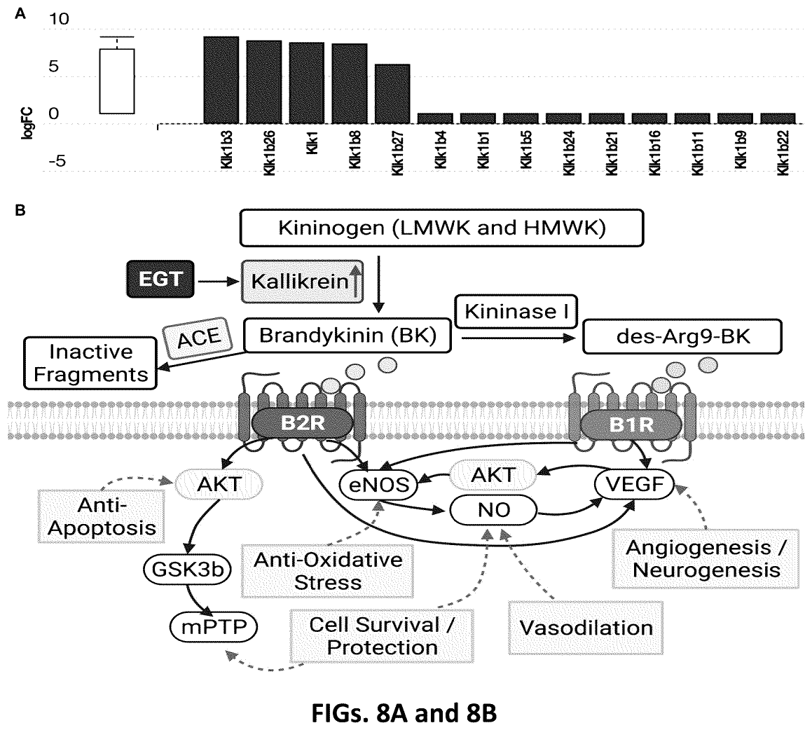

RNA-seq analysis indicates that ergothioneine treatment up-regulate pancreatic/renal kallikrein (Klk1) related Kallikrein system, which likely primarily contributes to the protect effects as discussed above (FIGS. 8). KLK1 is a key protein in the Kallikrein-Kinin system (KKS). The KKS promotes local blood flow and vasodilation, and may also reduce inflammation and oxidative stress. KLK1 is a serine protease that plays a critical role in regulating blood pressure, blood flow, and microcirculation. It's a secretory product of the salivary glands and the pancreas, and is also found in the kidney. KLK1 has a major function of reducing blood pressure by releasing the peptide hormone Lys-bradykinin (kallidin) from low molecular weight kininogen. This affects muscle relaxation and inflammatory processes. KLK1 also has anti-inflammatory, anti-apoptotic, anti-fibrotic, and anti-oxidative actions.

Other objects, features, and advantages of the present invention will become apparent from the following detailed description. It should be understood, however, that the detailed description and the examples, while indicating specific embodiments of the invention, are given by way of illustration only. Additionally, it is contemplated that changes and modifications within the spirit and scope of the invention will become apparent to those skilled in the art from this detailed description.

1. A method for modulating the lifespan of a cell, tissue, organ or organism, comprising contacting the cell, tissue, organ or organism with a composition comprising a therapeutically effective amount of a compound of Formula I, or a pharmaceutically acceptable prodrug, a pharmaceutically active metabolite, a pharmaceutically acceptable salt thereof, wherein the compound is represented by:

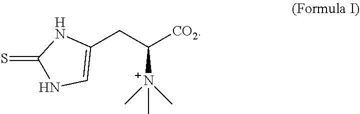

2. The method of claim 1, wherein modulating comprises increasing the level of activity of a gene having a nucleotide sequence of SEQ ID NOs.: 1-5.

3. The method of claim 1, wherein modulating comprises increasing the level of activity of a gene selected from the group consisting of DAF-16, FOXO1, FOX02, FOXO3, and FOX06.

4. The method of claim 1, wherein modulating comprises increasing the expression level of the gene.

5. The method of claim 4, wherein increasing the expression level of the gene comprises down-regulating any one of RAB-1, PMK-1, AKT-1, AKT2, JNK-1, PDK-1, and DAF-2.

6. The method of claim 1, wherein the cell exists in an in vitro condition.

7. The method of claim 1, wherein the organism is a mammal.

8. The method of claim 1, wherein the composition is administered intratumorally, intravenously, subcutaneously, intraosseously, orally, transdermally, in sustained release, in controlled release, in delayed release, as a suppository, or sublingually.

9. A method for increasing the level of activity of a gene selected from the group consisting of DAF-16, FOXO1, FOXO2, FOXO3, and FOX06 in a cell, tissue, organ or organism, comprising contacting the cell, tissue, organ or organism with a composition comprising an effective amount of a compound of Formula I, or a pharmaceutically acceptable prodrug, a pharmaceutically active metabolite, a pharmaceutically acceptable salt thereof, wherein the compound is represented by:

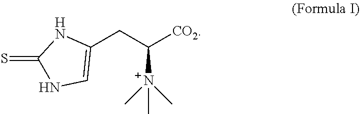

10. The method of claim 7, wherein the cell exists in an in vitro condition.

11. The method of claim 7, wherein the composition is administered intratumorally, intravenously, subcutaneously, intraosseously, orally, transdermally, in sustained release, in controlled release, in delayed release, as a suppository, or sublingually.

12. A method for modulating response or resistance to stress of a cell, tissue, organ or organism, comprising contacting the cell, tissue, organ or organism with a composition comprising an effective amount of the compound of Formula I, or an analog, a derivative, or a pharmaceutically acceptable prodrug, a pharmaceutically active metabolite, a pharmaceutically acceptable salt thereof, wherein the compound is represented by:

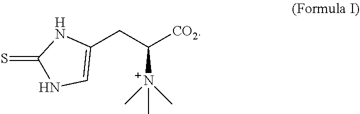

13. The method of claim 12, wherein the cell exists in an in vitro condition.

14. The method of claim 12, wherein the composition is administered intratumorally, intravenously, subcutaneously, intraosseously, orally, transdermally, in sustained release, in controlled release, in delayed release, as a suppository, or sublingually.

15. (canceled)

16. (canceled)

17. (canceled)

18. (canceled)

19. (canceled)


SEQUENCE LISTING (12 sequences)

SEQ ID NO: 1 - protein, 510 aa, Homo sapiens
MMEMLVDQGTDASSSASTSTSSVSRFGADTFMNTPDDVMMNDDMEPIPRDRCNTWPMRRPQLEPPLNSSPIIHEQIPEEDADLYGSNEQCGQLGGASSNGSTAMLHTPDGSNSHQTSFPSDFRMSESPDDTVSGKKTTTRRNAWGNMSYAELITTAIMASPEKRLTLAQVYEWMVQNVPYFRDKGDSNSSAGWKNSIRHNLSLHSRFMRIQNEGAGKSSWWVINPDAKPGRNPRRTRERSNTIETTTKAQLEKSRRGAKKRIKERALMGSLHSTLNGNSIAGSIQTISHDLYDDDSMQGAFDNVPSSFRPRTQSNLSIPGSSSRVSPAIGSDIYDDLEFPSWVGESVPAIPSDIVDRTDQMRIDATTHIGGVQIKQESKPIKTEPIAPPPSYHELNSVRGSCAQNPLLRNPIVPSTNFKPMPLPGAYGNYQNGGITPINWLSTSNSSPLPGIQSCGIVAAQHTVASSSALPIDLENLTLPDQPLMDTMDVDALIRHELSQAGGQHIHFDL

SEQ ID NO: 2 - protein, 655 aa, Homo sapiens
MAEAPQVVEIDPDFEPLPRPRSCTWPLPRPEFSQSNSATSSPAPSGSAAANPDAAAGLPSASAAAVSADFMSNLSLLEESEDFPQAPGSVAAAVAAAAAAAATGGLCGDFQGPEAGCLHPAPPQPPPPGPLSQHPPVPPAAAGPLAGQPRKSSSSRRNAWGNLSYADLITKAIESSAEKRLTLSQIYEWMVKSVPYFKDKGDSNSSAGWKNSIRHNLSLHSKFIRVQNEGTGKSSWWMLNPEGGKSGKSPRRRAASMDNNSKFAKSRSRAAKKKASLQSGQEGAGDSPGSQFSKWPASPGSHSNDDFDNWSTFRPRTSSNASTISGRLSPIMTEQDDLGEGDVHSMVYPPSAAKMASTLPSLSEISNPENMENLLDNLNLLSSPTSLTVSTQSSPGTMMQQTPCYSFAPPNTSLNSPSPNYQKYTYGQSSMSPLPQMPIQTLQDNKSSYGGMSQYNCAPGLLKELLTSDSPPHNDIMTPVDPGVAQPNSRVLGQNVMMGPNSVMSTYGSQASHNKMMNPSSHTHPGHAQQTSAVNGRPLPHTVSTMPHTSGMNRLTQVKTPVQVPLPHPMQMSALGGYSSVSSCNGYGRMGLLHQEKLPSDLDGMFIERLDCDMESIIRNDLMDGDTLDFNFDNVLPNQSFPHSVKTTTHSWVSG

SEQ ID NO: 3 - protein, 673 aa, Homo sapiens
MAEAPASPAPLSPLEVELDPEFEPQSRPRSCTWPLQRPELQASPAKPSGETAADSMIPEEEDDEDDEDGGGRAGSAMAIGGGGGSGTLGSGLLLEDSARVLAPGGQDPGSGPATAAGGLSGGTQALLQPQQPLPPPQPGAAGGSGQPRKCSSRRNAWGNLSYADLITRAIESSPDKRLTLSQIYEWMVRCVPYFKDKGDSNSSAGWKNSIRHNLSLHSRFMRVQNEGTGKSSWWIINPDGGKSGKAPRRRAVSMDNSNKYTKSRGRAAKKKAALQTAPESADDSPSQLSKWPGSPTSRSSDELDAWTDFRSRTNSNASTVSGRLSPIMASTELDEVQDDDAPLSPMLYSSSASLSPSVSKPCTVELPRLTDMAGTMNLNDGLTENLMDDLLDNITLPPSQPSPTGGLMQRSSSFPYTTKGSGLGSPTSSFNSTVFGPSSLNSLRQSPMQTIQENKPATFSSMSHYGNQTLQDLLTSDSLSHSDVMMTQSDPLMSQASTAVSAQNSRRNVMLRNDPMMSFAAQPNQGSLVNQNLLHHQHQTQGALGGSRALSNSVSNMGLSESSSLGSAKHQQQSPVSQSMQTLSDSLSGSSLYSTSANLPVMGHEKFPSDLDLDMFNGSLECDMESIIRSELMDADGLDFNFDSLISTQNVVGLNVGNFTGAKQASSQSWVPG

SEQ ID NO: 4 - protein, 673 aa, Homo sapiens; identical to SEQ ID NO: 3

SEQ ID NO: 5 - protein, 559 aa, Homo sapiens
MAAKLRAHQVDVDPDFAPQSRPRSCTWPLPQPDLAGDEDGALGAGVAEGAEDCGPERRATAPAMAPAPPLGAEVGPLRKAKSSRRNAWGNLSYADLITKAIESAPDKRLTLSQIYDWMVRYVPYFKDKGDSNSSAGWKNSIRHNLSLHTRFIRVQNEGTGKSSWWMLNPEGGKTGKTPRRRAVSMDNGAKFLRIKGKASKKKQLQAPERSPDDSSPSAPAPGPVPAAAKWAASPASHASDDYEAWADFRGGGRPLLGEAAELEDDEALEALAPSSPLMYPSPASALSPALGSRCPGELPRLAELGGPLGLHGGGGAGLPEGLLDGAQDAYGPRARAGTPAYFGGCKGGAYGGGGGFGPPAMGALRRLPMQTIQENKQASFVPAAAPFRPGALPALLPPPPPAPRPGPVLGAPGELALAGAAAAYPGKGAAPYAPPAPSRSALAHPISLMTLPGEAGAAGLAPSGHAAAFGGPPGGLLLDALPGPYAAAAAGPLGAAPDRFPADLDLDMFSGSLECDVESIILNDFMDSDEMDFNFDSALPPPPPGLAGAPPPNQSWVPG

SEQ ID NO: 6 - protein, 204 aa, Caenorhabditis elegans
AAMNPEYDYLFKLLLIGDSGVGKSCLLLRFADDTYTESYISTIGVDFKIRTIELDGKTIKLQIWDTAGQERFRTITSSYYRGAHGIIVVYDITDQETFNNVKQWLQEIDRYACENVNKLLVGNKCDLTAKRAVETQAAQDYAGQLGIPFLETSAKSSTNVEQAFLTMASEIKSRMGPVQGAGGAPGVRITGSQPVQDKKSGGCC

SEQ ID NO: 7 - protein, 377 aa, Caenorhabditis elegans
MFPQTTMDHILHPTPREGYYVVELNRSVWVVPNYYINLTPIGTGAYGTVCAAECTRSGTRVAIKKFNRPFQSIIHARRTYRELRLLRCMCHENIIDLLDVFTPNENVNDIEDVYFVSMLMGADLSNILKIQRLNDDHIQFLVYQILRGLKYIHSADIIHRDLKPSNIAVNEDCELKILDFGLARQTDSEMTGYVATRWYRAPEIMLNWMHYTQTVDVWSVGCILAELITGKTLFPGSDHIDQLTRIMSVTGTPDEEFLKKISSEEARNYIRNLPKMTRRDFKRLFAQATPQAIDLLEKMLHLDPDRRPTAKEAMEHEYLAAYHDETDEPIAEEMDLNDDVRADTIDEWKKIIWEEISDFQKNVAFADEEEDEEKMES

SEQ ID NO: 8 - protein, 254 aa, Caenorhabditis elegans
MSMTSLSTKSRRQEDVVIEGWLHKKGEHIRNWRPRYFMIFNDGALLGFRAKPKEGQPFPEPLNDFMIKDAATMLFEKPRPNMFMVRCLQWTTVIERTFYAESAEVRQRWIHAIESISKKYKGTNANPQEELMETNQQPKIDEDSEFAGAAHAIMGQPSSGHGDNCSIDFRASMISIADTSEAAKRDKITMEDFDFLKVLGKGTFGKVILCKEKRTQKLYAIKILKKDVIIAREEVAHTLTENRVLQRCKHPFLT

SEQ ID NO: 9 - protein, 483 aa, Caenorhabditis elegans
MSTENAHLQKEDIVIESWLHKKGEHIRNWRPRYFILFRDGTLLGFRSKPKEDQPLPEPLNNFMIRDAATVCLDKPRPNMFIVRCLQWTTVIERTFYADSADFRQMWIEAIQAVSSHNRLKENAGNTSMQEEDTNGNPSGESDVNMDATSTRSDNDFESTVMNIDEPEEVPRKNTVTMDDFDFLKVLGQGTFGKVILCREKSSDKLYAIKIIRKEMVVDRSEVAHTLTENRVLYACVHPFLTLLKYSFQAQYHICFVMEFANGGELFTHLQRCKTFSEARTRFYGSEIILALGYLHHRNIVYRDMKLENLLLDRDGHIKITDFGLCKEEIKYGDKTSTFCGTPEYLAPEVIEDIDYDRSVDWWGVGVVMYEMMCGRLPFSAKENGKLFELITTCDLKFPNRLSPEAVTLLSGLLERVPAKRLGAGPDDAREVSRAEFFKDVDWEATLRKEVEPPFKPNVMSETDTSFFDRVRYVSILLKVSEAI

SEQ ID NO: 10 - protein, 551 aa, Homo sapiens
MISPDPRPSPGLARWAESYEAKCERRQEIRESRRCRPNVTTCRQVGKTLRIQQREQLQRARLQQFFRRRNLELEEKGKAQHPQAREQGPSRRPGQVTVLKEPLSCARRISSPREQVTGTSSEVFPAQHPPPSGICRDLSDHLSSQAGGLPPQDTPIKKPPKHHRGTQTKAEGPTIKNDASQQTNYGVAVLDKEIIQLSDYLKEALQRELVLKQKMVILQDLLSTLIQASDSSWKGQLNEDKLKGKLRSLENQLYTCTQKYSPWGMKKVLLEMEDQKNSYEQKAKESLQKVLEEKMNAEQQLQSTQRSLALAEQKCEEWRSQYEALKEDWRTLGTQHRELESQLHVLQSKLQGADSRDLQMNQALRFLENEHQQLQAKIECLQGDRDLCSLDTQDLQDQLKRSEAEKLTLVTRVQQLQGLLQNQSLQLQEQEKLLTKKDQALPVWSPKSFPNEVEPEGTGKEKDWDLRDQLQKKTLQLQAKEKECRELHSELDNLSDEYLSCLRKLQHCREELNQSQQLPPRRQCGRWLPVLMVVIAAALAVFLANKDNLMI

SEQ ID NO: 11 - protein, 407 aa, Homo sapiens
MRWVWALLKNASLAGAPKYIEHFSKFSPSPLSMKQFLDFGSSNACEKTSFTFLRQELPVRLANIMKEINLLPDRVLSTPSVQLVQSWYVQSLLDIMEFLDKDPEDHRTLSQFTDALVTIRNRHNDVVPTMAQGVLEYKDTYGDDPVSNQNIQYFLDRFYLSRISIRMLINQHTLIFDGSTNPAHPKHIGSIDPNCNVSEVVKDAYDMAKLLCDKYYMASPDLEIQEINAANSKQPIHMVYVPSHLYHMLFELFKNAMRATVESHESSLILPPIKVMVALGEEDLSIKMSDRGGGVPLRKIERLFSYMYSTAPTPQPGTGGTPLAGFGYGLPISRLYAKYFQGDLQLFSMEGFGTDAVIYLKALSTDSVERLPVYNKSAWRHYQTIQEAGDWCVPSTEPKNTSTYRVS

SEQ ID NO: 12 - protein, 1846 aa, Caenorhabditis elegans
MTRMNIVRCRRRHKILENLEEENLGPSCSSTTSTTAATEALGTTTEDMRLKQQRSSSRATEHDIVDGNHHDDEHITMRRLRLVKNSRTRRRTTPDSSMDCYEENPPSQKTSINYSWISKKSSMTSLMLLLLFAFVQPCASIVEKRCGPIDIRNRPWDIKPQWSKLGDPNEKDLAGQRMVNCTVVEGSLTISFVLKHKTKAQEEMHRSLQPRYSQDEFITFPHLREITGTLLVFETEGLVDLRKIFPNLRVIGGRSLIQHYALIIYRNPDLEIGLDKLSVIRNGGVRIIDNRKLCYTKTIDWKHLITSSINDVVVDNAAEYAVTETGLMCPRGACEEDKGESKCHYLEEKNQEQGVERVQSCWSNTTCQKSCAYDRLLPTKEIGPGCDANGDRCHDQCVGGCERVNDATACHACKNVYHKGKCIEKCDAHLYLLLQRRCVTREQCLQLNPVLSNKTVPIKATAGLCSDKCPDGYQINPDDHRECRKCVGKCEIVCEINHVIDTFPKAQAIRLCNIIDGNLTIEIRGKQDSGMASELKDIFANIHTITGYLLVRQSSPFISLNMFRNLRRIEAKSLFRNLYAITVFENPNLKKLFDSTTDLTLDRGTVSIANNKMLCFKYIKQLMSKLNIPLDPIDQSEGTNGEKAICEDMAINVSITAVNADSVFFSWPSFNITDIDQRKFLGYELFFKEVPRIDENMTIEEDRSACVDSWQSVFKQYYETSNGEPTPDIFMDIGPRERIRPNTLYAYYVATQMVLHAGAKNGVSKIGFVRTSYYTPDPPTLALAQVDSDAIHITWEAPLQPNGDLTHYTIMWRENEVSPYEEAEKFCTDASTPANRQRTKDPKETIVADKPVDIPSSRTVAPTLLTMMGHEDQQKTCAATPGCCSCSAIEESSEQNKKKRPDPMSAIESSAFENKLLDEVLMPRDTMRVRRSIEDANRVSEELEKAENLGKAPKTLGGKKPLIHISKKKPSSSSTTSTPAPTIASMYALTRKPTTVPGTRIRLYEIYEPLPGSWAINVSALALDNSYVIRNLKHYTLYAISLSACQNMTVPGASCSISHRAGALKRTKHITDIDKVLNETIEWRFMNNSQQVNVTWDPPTEVNGGIFGYVVKLKSKVDGSIVMTRCVGAKRGYSTRNQGVLFQNLADGRYFVSVTATSVHGAGPEAESSDPIVVMTPGFFTVEIILGMLLVFLILMSIAGCIIYYYIQVRYGKKVKALSDFMQLNPEYCVDNKYNADDWELRQDDVVLGQQCGEGSFGKVYLGTGNNVVSLMGDRFGPCAIKINVDDPASTENLNYLMEANIMKNFKTNFIVQLYGVISTVQPAMVVMEMMDLGNLRDYLRSKREDEVFNETDCNFFDIIPRDKFHEWAAQICDGMAYLESLKFCHRDLAARNCMINRDETVKIGDFGMARDLFYHDYYKPSGKRMMPVRWMSPESLKDGKFDSKSDVWSFGVVLYEMVTLGAQPYIGLSNDEVLNYIGMARKVIKKPECCENYWYKVMKMCWRYSPRDRPTFLQLVHLLAAEASPEFRDLSFVLTDNQMILDDSEALDLDDIDDTDMNDQVVEVAPDVENVEVQSDSERRNTDSIPLKQFKTIPPINATTSHSTISIDETPMKAKQREGSLDEEYALMNHSGGPSDAEVRTYAGDGDYVERDVRENDVPTRRNTGASTSSYTGGGPYCLTNRGGSNERGAGFGEAVRLTDGVGSGHLNDDDYVEKEISSMDTRRSTGASSSSYGVPQTNWSGNRGATYYTSKAQQAATAAAAAAAALQQQQNGGRGDRLTQLPGTGHLQSTRGGQDGDYIETEPKNYRNNGSPSRNGNSRDIFNGRSAFGENEHLIEDNEHHPLV